(19) United States
(12) Patent Application Publication
(10) Pub. No.: US 2015/0030666 A1
(43) Pub. Date: Jan. 29, 2015

(54) ADHESIVE SKIN PATCH

(71) Applicant: KM TRANSDERM LTD., Osaka-shi, Osaka (JP)

(72) Inventors: Hiroyuki Ogino, Higashikagawa-shi (JP); Masaoki Goto, Higashikagawa-shi (JP); Atsuyo Hamada, Higashikagawa-shi (JP); Mitsuji Akazawa, Higashikagawa-shi (JP)

(21) Appl. No.: 14/360,831

(22) PCT Filed: Nov. 28, 2012
(86) PCT No.: PCT/JP2012/080767

§ 371 Date: Sep. 24, 2014

(30) Foreign Application Priority Data
Nov. 28, 2011 (JP) 2011-259523
Feb. 10, 2012 (JP) 2012-027739
Mar. 7, 2012 (JP) 2012-072354
Mar. 7, 2012 (JP) 2012-072358

(51) Int. Cl. A61K 9/70; A61K 31/4725; A61K 31/137; A61K 31/4025

(52) U.S. Cl. CPC A61K 9/7053; A61K 31/4025; A61K 31/4725; A61K 31/137
USPC 424/449; 514/422; 514/305; 514/648

(57) ABSTRACT

The present invention provides an adhesive skin patch containing a support and an adhesive layer containing a drug which is formed on the support, wherein the adhesive layer comprises a thermoplastic elastomer, a liquid component in an amount exceeding 300 parts by weight per 100 parts by weight of the thermoplastic elastomer, a therapeutic drug having an anticholinergic activity or a salt thereof for overactive bladder as a drug, (A) a non-volatile hydrocarbon oil as a liquid component, and one or more kinds selected from the group consisting of (B) an amide solvent, (C) an alcohol solvent and (D) a liquid organic acid as a liquid component, or a fatty acid salt, and optionally comprises a tackifier at a content of not more than 10 wt % of the adhesive layer.

### TECHNICAL FIELD

The present invention relates to an adhesive skin patch containing a therapeutic drug for overactive bladder, which has an anticholinergic activity, or a salt thereof. More particularly, the present invention relates to an adhesive skin patch showing high skin permeability of a therapeutic drug for overactive bladder, which has an anticholinergic activity, or a salt thereof, and good transdermal absorbability.

### BACKGROUND ART

Overactive bladder is a syndrome essentially associated with an urge to urinate, which is generally defined to accompany frequent urination and nocturia but does not essentially accompany urge incontinence (non-patent document 1). It is assumed that 1 out of 8 men and women at the age of 40 years or older has a symptom of overactive bladder, and the ratio becomes higher as the age increases. The characteristics of this symptom are that it can be developed regardless of gender, it not only degrades the quality of life but also forces a large societal burden in terms of care, well-being and economical aspect.

Various anticholinergic agents are generally used for the treatment of overactive bladder by orally administering them in the form of a salt.

For example, solifenacin, i.e., (3R)-1-azabicyclo[2.2.2]oct-3-yl(1S)-1-phenyl-3,4-dihydroisoquinoline-2(1H)-carboxylate, is generally used as solifenacin succinate for the treatment of overactive bladder. It is shown that solifenacin succinate is used for oral administration to an adult at 5 mg once per day (maximum 10 mg per day), and is a highly superior medicament since its half-life in blood is as long as about 50 hr.

Darifenacin, i.e., (3S)-1-[2-[(2,3-dihydrobenzofuran)-5-yl]ethyl]-α,α-diphenyl-3-pyrrolidineacetamide, is generally used as darifenacin hydrobromide for the treatment of overactive bladder. It is shown that darifenacin hydrobromide is used for oral administration to an adult at 7.5 mg once per day (maximum 15 mg per day), and is a highly superior medicament since side effects are less as compared to conventional anticholinergic agents.

Tolterodine, i.e., 4-methyl-2-[(R)-3-(diisopropylamino)-1-phenylpropyl]phenol, is generally used as tolterodine tartrate for the treatment of overactive bladder. It is shown that tolterodine tartrate is used for oral administration to an adult at 4.0 mg once per day, and is a highly superior medicament since side effects are less as compared to conventional anticholinergic agents.

However, the number of elderly persons in need of care such as bedridden patients, dementia patients and the like is increasing in recent years when aging has advanced. These elderly persons often have difficulty in swallowing, and administration of oral preparations to be taken regularly is often difficult. In patent document 1, therefore, exploitation of a new administration method of a muscarinic receptor antagonist is disclosed, and provision of a transdermal therapeutic system (TTS) other than oral administration was tried; however, the effect afforded by applying the above-mentioned 3 kinds of medicaments as an adhesive skin patch is not described.

When a drug is transdermally absorbed in TTS, the drug is added to an adhesive base and the like to form an adhesive skin patch. In recent years, tapes superior in adhesiveness to poultice containing a large amount of water as a constituent component in an adhesive skin patch are often used. As an adhesive base of the tapes, a lipophilic adhesive base such as a rubber adhesive base, an acrylic adhesive base, a silicone adhesive base and the like is used. Of these, a rubber adhesive base is widely used since additives can be easily blended as compared to other adhesive bases (patent documents 2-4).

### DOCUMENT LIST

### Patent Documents

patent document 1: WO 2005/011683
patent document 2: JP-A-2001-302502
patent document 3: JP-A-9-291028
patent document 4: JP-A-10-316559

### Non-Patent Document

Non-Patent Document 1: KAKATSUDOUBOUKOU SHINNRYOU GUIDELINE (overactive bladder treatment guideline), NIHONN HAINYOUKINOU GAKKAI KAKATSUDOUBOUKOU GUIDELINE SAKUSEI IINNKAI (The Japan Neurogenic Bladder Society overactive bladder guideline preparation committee) ed., p. 2-5, Blackwell Publishing, 2005

### SUMMARY OF THE INVENTION

### Problems to be Solved by the Invention

In an attempt to develop an adhesive skin patch composed of the adhesive bases described in, for example, patent documents 2-4 and containing a medicament, the present inventors have studied an adhesive skin patch containing, as a medicament, a therapeutic drug having an anticholinergic activity or a salt thereof for overactive bladder. However, it was clarified that an adhesive skin patch using a rubber adhesive base cannot ensure sufficient drug releasability, or skin irritation occurs due to a tackifier generally added to an adhesive skin patch.

In view of the above-mentioned problems and the like, an object of the present invention is to provide an adhesive skin patch having sufficient adhesiveness and low skin irritation, showing good skin permeability of a therapeutic drug having an anticholinergic activity or a salt thereof for overactive bladder, and showing sufficient transdermal absorbability.

### Means of Solving the Problems

The present inventors have conducted intensive studies in an attempt to solve the aforementioned problems and successfully afforded sufficient adhesiveness and reduced skin irritation by using, as an adhesive base, a thermoplastic elastomer and a large amount of a liquid component relative to the elastomer, and reducing the amount of a tackifier. They have further found that an adhesive skin patch of a therapeutic drug having an anticholinergic activity or a salt thereof for overactive bladder, which shows good skin permeability and sufficient transdermal absorbability, can be obtained by adding one or more kinds selected from the group consisting of alcohol solvent and liquid organic acid as a liquid component, or a fatty acid salt, together with a non-volatile hydrocarbon oil and an amide solvent as a liquid component, when the therapeutic drug having an anticholinergic activity or a salt thereof for overactive bladder is contained. The present invention based on this finding is as described below.

[1] An adhesive skin patch comprising a support and an adhesive layer containing a drug which is formed on the support, wherein

the adhesive layer comprises

a thermoplastic elastomer,

a liquid component in an amount exceeding 300 parts by weight per 100 parts by weight of the thermoplastic elastomer,

a therapeutic drug having an anticholinergic activity or a salt thereof for overactive bladder as a drug,

(A) a non-volatile hydrocarbon as a liquid component, and

one or more kinds selected from the group consisting of (B) an amide solvent, (C) an alcohol solvent and (D) a liquid organic acid as a liquid component, or a fatty acid salt, and optionally comprises a tackifier at a content of not more than wt % of the adhesive layer.

[2] The adhesive skin patch of the aforementioned [1], wherein the therapeutic drug having an anticholinergic activity for overactive bladder is one or more kinds selected from the group consisting of solifenacin, darifenacin and tolterodine.
[3] The adhesive skin patch of the aforementioned [1] or [2], wherein the adhesive layer comprises, as the liquid component, (B) an amide solvent and one or more kinds selected from the group consisting of (C) an alcohol solvent and (D) a liquid organic acid, and

the total content of (B) an amide solvent and one or more kinds selected from the group consisting of (C) an alcohol solvent and (D) a liquid organic acid is 10 wt %-60 wt % of the liquid component.

[4] The adhesive skin patch of any one of the aforementioned [1]-[3], wherein the adhesive layer comprises, as the liquid component, (B) an amide solvent and (C) an alcohol solvent.
[5] The adhesive skin patch of the aforementioned [4], wherein the total content of (B) an amide solvent and (C) an alcohol solvent is 10 wt %-60 wt % of the liquid component.
[6] The adhesive skin patch of any one of the aforementioned [1]-[5], wherein the adhesive layer further comprises, as the liquid component, (E) an ester solvent.
[7] The adhesive skin patch of any one of the aforementioned [1]-[6], wherein (A) a non-volatile hydrocarbon oil is liquid paraffin.
[8] The adhesive skin patch of any one of the aforementioned [1]-[7], wherein the fatty acid salt is a fatty acid salt having 12 or more carbon atoms.
[9] The adhesive skin patch of the aforementioned [8], wherein the adhesive layer comprises sodium oleate as at least one of the fatty acid salts.
[10] The adhesive skin patch of any one of the aforementioned [1]-[9], wherein the adhesive layer further contains a surfactant.
[11] The adhesive skin patch of the aforementioned [10], wherein the surfactant is sorbitan fatty acid ester.
[12] The adhesive skin patch of any one of the aforementioned [1]-[11], wherein the content of the liquid component in the adhesive layer is not less than 50 wt %.
[13] The adhesive skin patch of any one of the aforementioned [1]-[12], wherein the thermoplastic elastomer is a styrene block copolymer.
[14] The adhesive skin patch of the aforementioned [13], wherein the styrene block copolymer is one or more kinds selected from the group consisting of a styrene-isoprene-styrene block copolymer and a styrene-isoprene block copolymer.
[15] The adhesive skin patch of any one of the aforementioned [1]-[14], wherein the adhesive layer does not contain a tackifier.

A preferable embodiment of the present invention using solifenacin or a salt thereof (hereinafter sometimes to be abbreviated as “embodiment 1”) is as described below.

[1a] An adhesive skin patch comprising a support and an adhesive layer containing a drug which is formed on the support, wherein

the adhesive layer comprises

a thermoplastic elastomer,

a liquid component in an amount exceeding 300 parts by weight per 100 parts by weight of the thermoplastic elastomer,

solifenacin or a salt thereof as a drug,

(A) a non-volatile hydrocarbon oil as a liquid component, and

one or more kinds selected from the group consisting of (B) an amide solvent and (C) an alcohol solvent as a liquid component, and optionally comprises a tackifier at a content of not more than 10 wt % of the adhesive layer.

[2a] The adhesive skin patch of the aforementioned [1a], wherein the total content of one or more kinds selected from the group consisting of (B) an amide solvent and (C) an alcohol solvent is 10 wt %-60 wt % of the liquid component.
[3a] The adhesive skin patch of the aforementioned [1a] or [2a], wherein the adhesive layer further comprises, as the liquid component, (E) an ester solvent.
[4a] The adhesive skin patch of any one of the aforementioned [1a]-[3a], wherein (A) a non-volatile hydrocarbon oil is liquid paraffin.
[5a] The adhesive skin patch of any one of the aforementioned [1a]-[4a], wherein the adhesive layer further contains a surfactant.
[6a] The adhesive skin patch of the aforementioned [5a], wherein the surfactant is sorbitan fatty acid ester.
[7a] The adhesive skin patch of any one of the aforementioned [1a]-[6a], wherein the content of the liquid component in the adhesive layer is not less than 50 wt %.
[8a] The adhesive skin patch of any one of the aforementioned [1a]-[7a], wherein the thermoplastic elastomer is a styrene block copolymer.
[9a] The adhesive skin patch of the aforementioned [8a], wherein the styrene block copolymer is one or more kinds selected from the group consisting of a styrene-isoprene-styrene block copolymer and a styrene-isoprene block copolymer.
[10a] The adhesive skin patch of any one of the aforementioned [1a]-[9a], wherein the adhesive layer does not contain a tackifier.

A preferable embodiment of the present invention using darifenacin or a salt thereof (hereinafter sometimes to be abbreviated as “embodiment 2”) is as described below.

[1b] An adhesive skin patch comprising a support and an adhesive layer containing a drug which is formed on the support, wherein

the adhesive layer comprises

a thermoplastic elastomer,

a liquid component in an amount exceeding 300 parts by weight per 100 parts by weight of the thermoplastic elastomer,

darifenacin or a salt thereof as a drug,

(A) a non-volatile hydrocarbon oil, (B) an amide solvent and (D) a liquid organic acid as a liquid component, and optionally comprises a tackifier at a content of not more than 10 wt % of the adhesive layer.

[2b] The adhesive skin patch of the aforementioned [1b], wherein the total content of (B) an amide solvent and (D) a liquid organic acid is 10 wt %-60 wt % of the liquid component.
[3b] The adhesive skin patch of the aforementioned [1b] or [2b], wherein the adhesive layer further comprises, as the liquid component, (E) an ester solvent.
[4b] The adhesive skin patch of any one of the aforementioned [1b]-[3b], wherein (A) a non-volatile hydrocarbon oil is liquid paraffin.
[5b] The adhesive skin patch of any one of the aforementioned [1b]-[4b], wherein the adhesive layer further contains a surfactant.
[6b] The adhesive skin patch of the aforementioned [5b], wherein the surfactant is sorbitan fatty acid ester.
[7b] The adhesive skin patch of any one of the aforementioned [1b]-[6b], wherein the content of the liquid component in the adhesive layer is not less than 50 wt %.
[8b] The adhesive skin patch of any one of the aforementioned [1b]-[7b], wherein the thermoplastic elastomer is a styrene block copolymer.
[9b] The adhesive skin patch of the aforementioned [8b], wherein the styrene block copolymer is one or more kinds selected from the group consisting of a styrene-isoprene-styrene block copolymer and a styrene-isoprene block copolymer.
[10b] The adhesive skin patch of any one of the aforementioned [1b]-[9b], wherein the adhesive layer does not contain a tackifier.

Another preferable embodiment of the present invention using darifenacin or a salt thereof (hereinafter sometimes to be abbreviated as “embodiment 3”) is as described below.

[1c] An adhesive skin patch comprising a support and an adhesive layer containing a drug which is formed on the support, wherein

the adhesive layer comprises

a thermoplastic elastomer,

a liquid component in an amount exceeding 300 parts by weight per 100 parts by weight of the thermoplastic elastomer,

darifenacin or a salt thereof as a drug,

a fatty acid salt,

(A) a non-volatile hydrocarbon oil and (B) an amide solvent as a liquid component, and optionally comprises a tackifier at a content of not more than 10 wt % of the adhesive layer.

[2c] The adhesive skin patch of the aforementioned [1c], wherein the content of (B) an amide solvent is 10 wt %-60 wt % of the liquid component.
[3c] The adhesive skin patch of the aforementioned [1c] or [2c], wherein the adhesive layer further comprises, as the liquid component, (E) an ester solvent.
[4c] The adhesive skin patch of the aforementioned [1c]-[3c], wherein the adhesive layer further comprises, as the liquid component, (C) an alcohol solvent.
[5c] The adhesive skin patch of any one of the aforementioned [1c]-[4c], wherein (A) a non-volatile hydrocarbon oil is liquid paraffin.
[6c] The adhesive skin patch of any one of the aforementioned [1c]-[5c], wherein the fatty acid salt is a fatty acid salt having 12 or more carbon atoms.
[7c] The adhesive skin patch of the aforementioned [6c], wherein the adhesive layer comprises sodium oleate as at least one of the fatty acid salts.
[8c] The adhesive skin patch of any one of the aforementioned [1c]-[7c], wherein the adhesive layer further contains a surfactant.
[9c] The adhesive skin patch of the aforementioned [8c], wherein the surfactant is sorbitan fatty acid ester.
[10c] The adhesive skin patch of any one of the aforementioned [1c]-[9c], wherein the content of the liquid component in the adhesive layer is not less than 50 wt %.
[11c] The adhesive skin patch of any one of the aforementioned [1c]-[10c], wherein the thermoplastic elastomer is a styrene block copolymer.
[12c] The adhesive skin patch of the aforementioned [11c], wherein the styrene block copolymer is one or more kinds selected from the group consisting of a styrene-isoprene-styrene block copolymer and a styrene-isoprene block copolymer.
[13c] The adhesive skin patch of any one of the aforementioned [1c]-[12c], wherein the adhesive layer does not contain a tackifier.

A preferable embodiment of the present invention using tolterodine or a salt thereof (hereinafter sometimes to be abbreviated as “embodiment 4”) is as described below.

[1d] An adhesive skin patch comprising a support and an adhesive layer containing a drug which is formed on the support, wherein

the adhesive layer comprises

a thermoplastic elastomer,

a liquid component in an amount exceeding 300 parts by weight per 100 parts by weight of the thermoplastic elastomer,

tolterodine or a salt thereof as a drug,

(A) a non-volatile hydrocarbon oil as a liquid component, and

one or more kinds selected from the group consisting of (B) an amide solvent and (D) a liquid organic acid as a liquid component, and optionally comprises a tackifier at a content of not more than 10 wt % of the adhesive layer.

[2d] The adhesive skin patch of the aforementioned [1d], wherein the total content of one or more kinds selected from the group consisting of (B) an amide solvent and (D) a liquid organic acid is 10 wt %-60 wt % of the liquid component.
[3d] The adhesive skin patch of the aforementioned [1d] or [2d], wherein the adhesive layer comprises, as the liquid component, (B) an amide solvent and (D) a liquid organic acid.
[4d] The adhesive skin patch of the aforementioned [3d], wherein the total content of (B) an amide solvent and (D) a liquid organic acid is 10 wt %-60 wt % of the liquid component.
[5d] The adhesive skin patch of any one of the aforementioned [1d]-[4d], wherein the adhesive layer further comprises, as the liquid component, (E) an ester solvent.
[6d] The adhesive skin patch of any one of the aforementioned [1d]-[5d], wherein (A) a non-volatile hydrocarbon oil is liquid paraffin.
[7d] The adhesive skin patch of any one of the aforementioned [1d]-[6d], wherein the adhesive layer further contains a surfactant.
[8d] The adhesive skin patch of the aforementioned [7d], wherein the surfactant is sorbitan fatty acid ester.
[9d] The adhesive skin patch of any one of the aforementioned [1d]-[8d], wherein the content of the liquid component in the adhesive layer is not less than 50 wt %.
[10d] The adhesive skin patch of any one of the aforementioned [1d]-[9d], wherein the thermoplastic elastomer is a styrene block copolymer.
[11d] The adhesive skin patch of the aforementioned [10d], wherein the styrene block copolymer is one or more kinds selected from the group consisting of a styrene-isoprene-styrene block copolymer and a styrene-isoprene block copolymer.
[12d] The adhesive skin patch of any one of the aforementioned [1d]-[11d], wherein the adhesive layer does not contain a tackifier.

### Effect of the Invention

The adhesive skin patch of the present invention shows good skin permeability of a therapeutic drug having an anticholinergic activity or a salt thereof for overactive bladder, has sufficient adhesiveness when adhered to the skin; and causes low skin irritation.

### DESCRIPTION OF EMBODIMENTS

In the adhesive skin patch of the present invention, the adhesive layer comprises

a thermoplastic elastomer,

(A) a non-volatile hydrocarbon as a liquid component, and

a therapeutic drug having an anticholinergic activity or a salt thereof for overactive bladder, and further

one or more kinds selected from the group consisting of (B) an amide solvent, (C) an alcohol solvent and (D) a liquid organic acid as a liquid component, or a fatty acid salt.

Each component that can be contained in the adhesive layer of the present invention may be only one kind or two or more kinds in combination.

In the adhesive skin patch of the present invention, the adhesive layer comprises, preferably,

a thermoplastic elastomer,

(A) a non-volatile hydrocarbon and (B) an amide solvent as a liquid component, and

a therapeutic drug having an anticholinergic activity or a salt thereof for overactive bladder, and further

one or more kinds selected from the group consisting of (C) an alcohol solvent and (D) a liquid organic acid as a liquid component, or a fatty acid salt.

When the adhesive layer contains, as the liquid component, (A) a non-volatile hydrocarbon oil, (B) an amide solvent, and one or more kinds selected from the group consisting of (C) an alcohol solvent and (D) a liquid organic acid, the total content of (B) an amide solvent, and one or more kinds selected from the group consisting of (C) an alcohol solvent and (D) a liquid organic acid is preferably 10 wt %-85 wt %, more preferably 10 wt %-60 wt %, most preferably 20 wt %-60 wt %, of the liquid component, to enhance the dispersibility and transdermal absorbability of the therapeutic drug having an anticholinergic activity or a salt thereof for overactive bladder in the adhesive layer.

The adhesive layer contains, preferably, as the liquid component, (A) a non-volatile hydrocarbon oil, (B) an amide solvent, and (C) an alcohol solvent. In this case, the total content of (B) an amide solvent, and (C) an alcohol solvent is preferably 10 wt %-85 wt %, more preferably 10 wt %-60 wt %, most preferably 20 wt %-60 wt %, of the liquid component, to enhance the dispersibility and transdermal absorbability of the therapeutic drug having an anticholinergic activity or a salt thereof for overactive bladder in the adhesive layer.

The therapeutic drug having an anticholinergic activity for overactive bladder is one or more kinds selected from the group consisting of solifenacin, darifenacin and tolterodine.

As a salt of solifenacin, acid addition salt of solifenacin with an organic acid, acid addition salt of solifenacin with an inorganic acid can be mentioned. Examples of the organic acid include monocarboxylic acids such as acetic acid, propionic acid, butyric acid and the like; dicarboxylic acids such as oxalic acid, malonic acid, fumaric acid, succinic acid, maleic acid and the like; hydroxycarboxylic acids such as hydroxyacetic acid, lactic acid, malic acid, citric acid, tartaric acid and the like; carbonic acid; alkane sulfonic acids such as methanesulfonic acid, ethanesulfonic acid and the like; amino acids such as glutamic acid and the like; and the like. Examples of the inorganic acid include hydrochloric acid, hydrobromic acid, sulfuric acid, nitric acid, phosphoric acid and the like. From the aspects of easy availability, dispersibility in the adhesive layer and the like, solifenacin succinate is preferable.

As a salt of darifenacin, acid addition salt of darifenacin with an organic acid, acid addition salt of darifenacin with an inorganic acid can be mentioned. Examples of the organic acid include monocarboxylic acids such as acetic acid, propionic acid, butyric acid and the like; dicarboxylic acids such as oxalic acid, malonic acid, fumaric acid, succinic acid, maleic acid and the like; hydroxycarboxylic acids such as hydroxyacetic acid, lactic acid, malic acid, citric acid, tartaric acid and the like; carbonic acid; alkane sulfonic acids such as methanesulfonic acid, ethanesulfonic acid and the like; amino acids such as glutamic acid and the like; and the like. Examples of the inorganic acid include hydrochloric acid, hydrobromic acid, sulfuric acid, nitric acid, phosphoric acid and the like. From the aspects of easy availability, dispersibility in the adhesive layer and the like, darifenacin hydrobromide is preferable.

As a salt of tolterodine, acid addition salt of tolterodine with an organic acid, acid addition salt of tolterodine with an inorganic acid can be mentioned. Examples of the organic acid include monocarboxylic acids such as acetic acid, propionic acid, butyric acid and the like; dicarboxylic acids such as oxalic acid, malonic acid, fumaric acid, succinic acid, maleic acid and the like; hydroxycarboxylic acids such as hydroxyacetic acid, lactic acid, malic acid, citric acid, tartaric acid and the like; carbonic acid; alkane sulfonic acids such as methanesulfonic acid, ethanesulfonic acid and the like; amino acids such as glutamic acid and the like; and the like. Examples of the inorganic acid include hydrochloric acid, hydrobromic acid, sulfuric acid, nitric acid, phosphoric acid and the like. From the aspects of easy availability, dispersibility in the adhesive layer and the like, tolterodine tartrate is preferable.

While the content of the therapeutic drug having an anticholinergic activity or a salt thereof for overactive bladder in the adhesive layer is not particularly limited, it is preferably 0.5 wt %-20 wt %, more preferably 1 wt %-17.5 wt %, most preferably 1 wt %-15 wt %, in consideration of the dispersibility in the adhesive layer and the transdermal absorbability.

In the present invention, the “thermoplastic elastomer” is an elastomer having thermoplasticity wherein it is softened when heat is added to show flowability, and returns to a rubbery elastic body when cooled, and examples thereof include various thermoplastic elastomers such as urethane thermoplastic elastomer, acrylic thermoplastic elastomer, styrene thermoplastic elastomer (for example, styrene block copolymer), olefin thermoplastic elastomer and the like. Of these, styrene thermoplastic elastomer, particularly styrene block copolymer, is preferable to simultaneously achieve sufficient adhesiveness and low skin irritation, which is the object of the present invention.

Specific examples of the styrene block copolymer include styrene-butadiene block copolymer, styrene-butadiene-styrene block copolymer, styrene-isoprene block copolymer, styrene-isoprene-styrene block copolymer, styrene-ethylene/butylene block copolymer, styrene-ethylene/butylene-styrene block copolymer, styrene-ethylene/propylene block copolymer, styrene-ethylene/propylene-styrene block copolymer, styrene-isobutylene block copolymer, styrene-isobutylene-styrene block copolymer and the like. In the above, “ethylene/butylene” shows an ethylene and butylene copolymer block, and “ethylene/propylene” shows an ethylene and propylene copolymer block. Only one kind of these styrene block copolymers may be used or two or more kinds thereof may be used in combination.

From the aspects of simultaneously achievement of sufficient adhesiveness and low skin irritation, and availability and handling property of the products for adhesive skin patch, of the above-mentioned styrene block copolymers, one or more kinds selected from the group consisting of a styrene-isoprene-styrene block copolymer and a styrene-isoprene block copolymer are particularly preferably used.

As the styrene-isoprene-styrene block copolymer, a copolymer having a styrene content of 5 wt %-60 wt % is preferable, and a copolymer having a styrene content of 10 wt %-50 wt % is more preferable. In addition, a copolymer having a weight average molecular weight as measured by gel filtration chromatography of 20,000-500,000 is preferable, and a copolymer having a weight average molecular weight of 30,000-300,000 is more preferable. As the styrene-isoprene block copolymer, a copolymer having a styrene content of 5 wt %-50 wt % is preferable, and a copolymer having a styrene content of 10 wt %-40 wt % is more preferable. In addition, a copolymer having a weight average molecular weight as measured by gel filtration chromatography of 10,000-500,000 is preferable, and a copolymer having a weight average molecular weight of 20,000-300,000 is more preferable.

As a styrene-isoprene-styrene block copolymer or styrene-isoprene block copolymer, a copolymer produced by a method known per se can also be used. A commercially available product that satisfies the above-mentioned properties, for example, “KRATON D” (manufactured by KRATON POLYMERS), “JSR SIS” (manufactured by JSR) and the like can also be used.

In the present invention, the “liquid component” means a component which is liquid at ambient temperature, does not volatilize during production and preservation and adhesion, remains in an adhesive layer, and is added to an adhesive skin patch as a plasticizer or softening agent, or a dispersing agent and/or a transdermal absorption promoter for a therapeutic drug having an anticholinergic activity or a salt thereof for overactive bladder. Therefore, the aforementioned liquid component is a substance having a melting point at a temperature lower than ambient temperature, and a boiling point of preferably not less than 150° 0, more preferably not less than 170° C. In the present invention, one having a certain level of viscosity can also be used. The “liquid component” of the present invention shows viscosity at 25° C. of 0.01 mPa s-1,000,000 mPa s. The “ambient temperature” is the ambient temperature (15° C.-25° C.) in The Japanese Pharmacopoeia Sixteenth Edition General Notices.

As (A) a non-volatile hydrocarbon oil, a chain saturated hydrocarbon having about 20-40 carbon atoms or a chain unsaturated hydrocarbon having about 20-40 carbon atoms is preferable and, for example, liquid paraffin, squalene, squalene, pristine and the like can be mentioned. In view of easy availability, liquid paraffin is more preferable. Liquid paraffin is a mixture of colorless odorless liquid alkane having not less than 20 carbon atoms. In the present invention, liquid paraffin compatible with the standard defined in the Japanese Pharmacopoeia, United States Pharmacopoeia and the like, and the like can be preferably used.

The content of (A) a non-volatile hydrocarbon oil in the liquid component is preferably 15 wt %-90 wt %, more preferably 40 wt %-90 wt %, further preferably 40 wt %-80 wt %, most preferably 40 wt %-70 wt %.

Examples of (B) an amide solvent include pyrrolidones such as N-methyl-2-pyrrolidone, 2-pyrrolidone and the like; imidazolidinones such as 1,3-dimethyl-2-imidazolidinone and the like; N-substituted toluidines such as crotamiton and the like; alkanamides such as formamide, N-methylformamide, N,N-dimethylformamide, N-methylacetamide, N,N-dimethylacetamide, N-methylpropanamide and the like; and the like.

Of the above-mentioned amide solvents, N-methyl-2-pyrrolidone, crotamiton, N,N-dimethylformamide and N,N-dimethylacetamide are preferable and N-methyl-2-pyrrolidone, crotamiton are more preferable to improve solubility, dispersibility and transdermal absorbability of a therapeutic drug having an anticholinergic activity or a salt thereof for overactive bladder.

Examples of (C) an alcohol solvent include higher saturated aliphatic alcohol having about 12-20 carbon atoms which is liquid at ambient temperature, such as lauryl alcohol, isostearyl alcohol, 2-octyldodecanol and the like; higher unsaturated aliphatic alcohol having about 12-20 carbon atoms which is liquid at ambient temperature, such as oleyl alcohol and the like; polyvalent alcohol which is liquid at ambient temperature such as ethylene glycol, propylene glycol, glycerol, 1,3-butanediol, polyethylene glycol having a molecular weight of about 100-600 and the like; and the like.

Of these, polyvalent alcohol which is liquid at ambient temperature such as ethylene glycol, propylene glycol, glycerol, 1,3-butanediol, polyethylene glycol and the like is preferable, diol which is liquid at ambient temperature such as ethylene glycol, propylene glycol, 1,3-butanediol, polyethylene glycol having a molecular weight of about 100-600 and the like are more preferable to improve solubility of a therapeutic drug having an anticholinergic activity or a salt thereof for overactive bladder.

Examples of (D) a liquid organic acid include aliphatic monocarboxylic acids such as acetic acid, propionic acid, butyric acid, valeric acid, isovaleric acid, caproic acid, enanthic acid(heptanoic acid), caprylic acid, pelargric acid(nonanoic acid) and the like; aliphatic unsaturated monocarboxylic acids such as oleic acid, linoleic acid, arachidonic acid, docosahexaenoic acid and the like; hydroxycarboxylic acids such as lactic acid (DL-lactic acid, D-lactic acid, L-lactic acid, mixture of DL-lactic acid and lactic anhydride, mixture of D-lactic acid and lactic anhydride, a mixture of L-lactic acid and lactic anhydride) and the like; alkoxy group-substituted liquid carboxylic acids such as methoxyacetic acid and the like; sulfonic acids such as methanesulfonic acid and the like; and the like.

These liquid organic acids have a function to aid dissolution of a therapeutic drug having an anticholinergic activity or a salt thereof for overactive bladder, as a result of which a therapeutic drug having an anticholinergic activity or a salt thereof for overactive bladder, which has low solubility, can be contained at a high concentration in an adhesive layer. They can also improve dispersibility, and further provide an effect of improving transdermal absorbability. From such aspect, lactic acid (particularly the Japanese Pharmacopoeia lactic acid) and oleic acid are preferably used, and lactic acid (particularly the Japanese Pharmacopoeia lactic acid) is more preferably used, from among these liquid organic acids.

To improve transdermal absorbability of a therapeutic drug having an anticholinergic activity or a salt thereof for overactive bladder, the above-mentioned liquid component preferably further contains (E) an ester solvent.

Examples of (E) an ester solvent include ester of long chain fatty acid and monovalent aliphatic alcohol, medium-chain triglyceride, ester of polyvalent carboxylic acid and monovalent aliphatic alcohol, carbonate and the like.

As an ester of long chain fatty acid and monovalent aliphatic alcohol, an ester, which is liquid at ambient temperature, of long chain saturated fatty acid having 12-20 carbon atoms and monovalent aliphatic alcohol having 1-20 carbon atoms is preferable, and examples thereof include myristate which is liquid at ambient temperature such as ethyl myristate, isopropyl myristate, octyldodecyl myristate and the like, palmitate which is liquid at ambient temperature such as ethyl palmitate, isopropyl palmitate, isostearyl palmitate and the like, stearate which is liquid at ambient temperature such as isopropyl stearate and the like, and the like. In addition, an ester of long-chain unsaturated fatty acid having 12-20 carbon atoms and monovalent aliphatic alcohol having 1-20 carbon atoms can also be used preferably, and examples thereof include oleate which is liquid at ambient temperature such as ethyl oleate, decyl oleate, oleyl oleate and the like, linoleate which is liquid at ambient temperature such as ethyl linoleate, isopropyl linoleate and the like, and the like.

Medium-chain triglyceride is a triglyceride of fatty acid having about 6-12 carbon atoms such as caproic acid, caprylic acid, capric acid, lauric acid and the like, and glycerol. In the present invention, caprylic acid triglyceride, a triglyceride mixture of caprylic acid and capric acid, a triglyceride mixture of caprylic acid, capric acid and lauric acid, and the like, which are liquid at ambient temperature, can be used. In addition, fats and oils containing a large amount of these, which are liquid at ambient temperature, can also be used. Examples of fats and oils include peanuts oil, olive oil, castor oil and the like.

As medium-chain triglyceride which is liquid at ambient temperature or medium-chain triglyceride containing fats and oils, which is liquid at ambient temperature, in the present invention, a commercially available product for pharmaceutical use can also be used.

Examples of the ester of polyvalent carboxylic acid and monovalent aliphatic alcohol include diester, which is liquid at ambient temperature, of dicarboxylic acid having 2-12 carbon atoms and monovalent aliphatic alcohol having 1-20 carbon atoms such as adipic acid diester which is liquid at ambient temperature such as diethyl adipate, diisopropyl adipate and the like, sebacic acid diester which is liquid at ambient temperature such as diethyl sebacate, diisopropyl sebacate, dioctyldodecyl sebacate and the like, and the like.

Examples of carbonate include cyclic carbonate of carbonic acid and diol having 2-10 carbon atoms, such as ethylene carbonate, propylene carbonate, vinylene carbonate and the like, with preference given to propylene carbonate.

Of the above-mentioned ester solvents, myristate, medium-chain triglyceride, sebacic acid diester and carbonate are preferable, isopropyl myristate, a triglyceride mixture of caprylic acid and capric acid, diethyl sebacate and propylene carbonate are more preferable.

Examples of fatty acid contained in the fatty acid salt include aliphatic monocarboxylic acid, alicyclic monocarboxylic acid, aliphatic dicarboxylic acid and the like.

Examples of aliphatic monocarboxylic acid include short chain fatty acids having 2-7 carbon atoms such as acetic acid, butyric acid, hexanoic acid and the like, middle chain fatty acids having 8-11 carbon atoms such as octanoic acid, decanoic acid and the like, long chain fatty acids having 12 or more carbon atoms such as myristic acid, stearic acid, isostearic acid, oleic acid and the like, hydroxymonocarboxylic acids such as glycolic acid, lactic acid, 3-hydroxybutyric acid, mandelic acid and the like, alkoxy group-substituted monocarboxylic acids such as methoxyacetic acid and the like, ketomonocarboxylic acids such as levulinic acid and the like, and the like.

Examples of alicyclic monocarboxylic acid include alicyclic monocarboxylic acids having 6-8 carbon atoms such as cyclohexane carboxylic acid and the like.

Examples of aliphatic dicarboxylic acid include sebacic acid, adipic acid, malic acid, maleic acid, fumaric acid and the like.

Preferable fatty acid includes, for example, fatty acid having 12 or more carbon atoms and hydroxymonocarboxylic acid, such as myristic acid, stearic acid, isostearic acid, oleic acid and lactic acid. More preferred are oleic acid and lactic acid.

Examples of cation contained in the fatty acid salt include alkali metal cations such as sodium ion, potassium ion and the like, alkaline earth metal cations such as calcium ion and the like, and N⁺(R)₃(H) wherein R is a hydrogen atom or organic group. From the aspects of easy availability, and the effect of improving stability and transdermal absorbability, the fatty acid salt is preferably fatty acid sodium.

In consideration of the effect of improving the stability of a drug and the effect of improving the transdermal absorbability of a drug, the fatty acid salt is preferably a fatty acid salt having 12 or more carbon atoms or hydroxymonocarboxylate, more preferably fatty acid sodium having 12 or more carbon atoms or sodium hydroxymonocarboxylate, further preferably sodium oleate or sodium lactate and particularly preferably sodium oleate. Only one kind of these may be used or two or more kinds thereof may be used in combination.

While the content of the fatty acid salt in an adhesive layer is not particularly limited, from the aspects of the transdermal absorbability of a drug and the property of an adhesive skin patch (e.g., adhesive property etc.), it is preferably not less than 0.1 mol and not more than 5 mol, more preferably not less than 0.2 mol and not more than 3 mol, per 1 mol of a therapeutic drug having an anticholinergic activity for overactive bladder.

To enhance the transdermal absorbability of a therapeutic drug having an anticholinergic activity or a salt thereof for overactive bladder, moreover, the adhesive layer of the adhesive skin patch of the present invention preferably further contains a surfactant.

Examples of the surfactant include non-ionic surfactants such as polyoxyethylene fatty acid esters such as polyoxyethylene monolaurate and the like, polyoxyethylene sorbit fatty acid esters such as polyoxyethylene sorbit tetraoleate and the like, polyoxyethylene sorbitan fatty acid esters such as polyoxyethylene sorbitan monooleate, polyoxyethylene sorbitan monolaurate, polyoxyethylene sorbitan monopalmitate and the like, sorbitan fatty acid esters such as sorbitan monolaurate, sorbitan monooleate, sorbitan sesquioleate, sorbitan trioleate and the like, fatty acid esters of glycerol such as glycerol monooleate, polyoxyethylene castor oil derivative, polyoxyethylene hydrogenated castor oil and the like, polyoxyethylene higher aliphatic alcohol ethers such as polyoxyethylene lauryl ether, polyoxyethylene oleyl ether and the like, polyoxyethylene alkyl phenyl ethers such as polyoxyethylene nonyl phenyl ether and the like, polyoxyethylene polyoxypropylene copolymer such as pluronic L-31, pluronic L-44 and the like, and the like, anionic surfactants such as sodium alkylsulfate (e.g., sodium lauryl sulfate and the like) and the like, cationic surfactants such as alkyl trimethyl ammonium salt, alkyl dimethyl ammonium salt and the like, amphoteric surfactants such as alkyl dimethyl amine oxide, alkylcarboxybetaine and the like.

Of these, to enhance the transdermal absorbability, a non-ionic surfactant which is liquid at ambient temperature is preferable, sorbitan fatty acid ester which is liquid at ambient temperature is more preferably, and sorbitan monolaurate is particularly preferable.

In the present invention, the content of the surfactant in the adhesive layer is preferably 0.01 wt %-10 wt %, more preferably 0.1 wt %-5 wt %.

The adhesive skin patch of the present invention contains a liquid component in an amount exceeding 300 parts by weight per 100 parts by weight of the thermoplastic elastomer. When the content of the liquid component relative to 100 parts by weight of the thermoplastic elastomer is not more than 300 parts by weight, sufficient adhesiveness cannot be achieved, whereas when the content of the liquid component relative to the thermoplastic elastomer is too much, the shape retention of the adhesive layer general becomes difficult. Therefore, the content of the liquid component does not generally exceed 1500 parts by weight per 100 parts by weight of the thermoplastic elastomer. The content of the liquid component is preferably 320 parts by weight-1000 parts by weight, more preferably 340 parts by weight-850 parts by weight, per 100 parts by weight the thermoplastic elastomer.

When the content of the thermoplastic elastomer in the adhesive layer is too small, the shape retention of the adhesive layer becomes difficult, when it is too much, adhesiveness becomes insufficient. Therefore, the thermoplastic elastomer content of the adhesive layer in the adhesive skin patch of the present invention is preferably 5 wt %-24.5 wt %, more preferably 8 wt %-24 wt %, particularly preferably 10 wt %-23.5 wt %. The content of the liquid component in the adhesive layer is preferably not less than 50 wt %, more preferably 50 wt %-87 wt %, further preferably 54.5 wt %-86 wt %, particularly preferably 60 wt %-86 wt %, most preferably 65 wt %-86 wt %.

The adhesive skin patch of the present invention can exhibit good adhesiveness, since the adhesive layer is composed of a thermoplastic elastomer and a liquid component at the above-mentioned contents and content ratios. The adhesive layer may contain a tackifier as necessary.

Here, the tackifier is a resin widely used for conferring adhesiveness generally in the field of adhesive skin patch, and examples thereof include rosin resin, polyterpene resin, coumarone-indene resin, petroleum resin, terpene-phenol resin, alicyclic saturated hydrocarbon resin and the like.

To decrease skin irritation and the like, the content of the tackifier in the adhesive layer is not more than 10 wt % in the present invention. The content is preferably not more than 5 wt %, more preferably not more than 2 wt %, further preferably not more than 1 wt %, and the absence of a tackifier in the adhesive layer is most preferable. In relation to the adhesiveness of the adhesive skin patch, the content of the tackifier is adjusted according to the kind, content and content ratio of the thermoplastic elastomer and liquid component.

The adhesive layer may further contain an optional component. Examples of the optional component include general additives such as excipient, dispersing agent, stabilizer, thickener, antioxidant, softening agent, flavoring agent, colorant and the like.

Examples of the excipient include silicon compound such as silicic anhydride, light anhydrous silicic acid, silicic hydride and the like; cellulose derivative such as ethylcellulose, methylcellulose, hydroxypropylcellulose, hydroxypropylmethylcellulose and the like; water-soluble synthesis polymer such as polyvinyl alcohol and the like; aluminum compound such as dried aluminum hydroxide gel, water-containing aluminum silicate and the like; pigment such as kaolin, titanium oxide and the like; and the like.

Examples of the dispersing agent include gum arabic, propyleneglycol alginate, sodium dioctyl sulfosuccinate, lecithin and the like.

Examples of the stabilizer include zinc stearate, gelatin, dextran, povidone and the like.

Examples of the thickener include carboxy vinyl polymer, tragacanth and the like.

Examples of the antioxidant include dibutylhydroxytoluene, ascorbic acid, ascorbyl stearate, tocopherol, tocopherol ester derivative (e.g., tocopherol acetate etc.), butylhydroxyanisole, 2-mercaptobenzimidazole, anthocyanin, catechin and the like.

Examples of the softening agent include fats and oils such as almond oil, rape seed oil, cottonseed oil-soybean oil mixture, process oil, beef tallow and the like; waxes such as purified lanolin and the like; esters which are solid at ambient temperature such as cetyl lactate and the like; rubbers such as polyisoprene rubber, polybutene, crude rubber and the like; polymer such as crystalline cellulose and the like; allantoin and the like.

Examples of the flavoring agent include d-camphor, dl-camphor, d-borneol, dl-borneol, cinnamaldehyde, peppermint oil, dl-menthol, 1-menthol and the like.

Examples of the colorant include red ferric oxide, yellow iron oxide, yellow ferric oxide, carbon black and the like.

From the aspects of improved stability and improved transdermal absorbability of a drug, the adhesive layer may contain lactone as an optional component.

Examples of the lactone include 5-membered ring lactone of ascorbic acid or a salt thereof (for example, sodium ascorbate), isoascorbic acid or a salt thereof (for example, sodium isoascorbate) and the like, and the like. While the content of lactone in the adhesive layer is not particularly limited from the aspects of the transdermal absorbability of a drug and the property of the adhesive skin patch (e.g., adhesive property etc.), it is preferably not less than 0.1 mol and not more than 5 mol, more preferably not less than 0.2 mol and not more than 3 mol, per 1 mol of a therapeutic drug having an anticholinergic activity for overactive bladder.

The adhesive skin patch of the present invention is prepared by casting an adhesive layer having the above-mentioned constitution on a support.

In the present invention, the “support” is not particularly limited, and one widely used for adhesive skin patches can be used. For example, woven fabric such as polyethylene woven fabric, polypropylene woven fabric and the like; non-woven fabric such as polyethylene non-woven fabric, polypropylene non-woven fabric and the like; film of polyethylene, polypropylene, polyester (poly(ethylene terephthalate) and the like), ethylene vinyl acetate copolymer, vinyl chloride and the like; foamed support such as urethane foamed support, polyurethane foamed support and the like; and the like can be mentioned. These may be used singly or a laminate of plural kinds may be used. Furthermore, to prevent accumulation of static electricity on the support, an antistatic agent may be added to the aforementioned woven fabric, non-woven fabric, film and the like constituting the support. Also, to provide good anchor property to the adhesive layer, non-woven fabric, woven fabric or a laminate thereof with a film can be used as a support. The thickness of a film as the support is generally 10 μm-100 μm, preferably 15 μm-50 μm, and the thickness of woven fabric, non-woven fabric, and a porous sheet such as foamed support and the like is generally 50 μm-2,000 μm, preferably 100 μm-1,000 μm.

In addition, the adhesive skin patch of the present invention can also be provided with a release liner generally used in the field of adhesive skin patches. As the release liner, glassine, polyethylene, polypropylene, polyester, poly(ethylene terephthalate), polystyrene, aluminum film, foamed polyethylene film, foamed polypropylene film and the like, or a laminate of two or more kinds of those mentioned above can be used. Moreover, these after silicone processing, fluorine resin processing, emboss processing, hydrophilic processing, hydrophobic processing and the like, and the like can also be used. The thickness of the release liner is generally 10 μm-200 μm, preferably 15 μm-150 μm.

The adhesive skin patch of the present invention can be produced by, for example, (1) dissolving a thermoplastic elastomer and a therapeutic drug having an anticholinergic activity or a salt thereof for overactive bladder in a liquid component, or (2) dissolving or dispersing a thermoplastic elastomer and a therapeutic drug having an anticholinergic activity or a salt thereof for overactive bladder in a solvent such as toluene and the like, preparing a coating solution for forming an adhesive layer, applying the obtained solution on a support and drying same. When a release liner is used, a release liner can be laminated by pressing same on an adhesive layer. Alternatively, the aforementioned solution is applied on a release liner, dried to form an adhesive layer on the surface of the release liner, and a support may be adhered by pressing same against the adhesive layer. A coating solution for forming an adhesive layer can be applied using, for example, a conventionally-used coater such as roll coater, die coater, gravure roll coater, reverse roll coater, kiss-roll coater, dip roll coater, bar coater, knife coater, spray coater and the like. In addition, the aforementioned solution is preferably dried under heating at, for example, about 40° C.-150° C. The adhesive layer after drying, which contains a therapeutic drug having an anticholinergic activity or a salt thereof for overactive bladder is preferably 10 g/m²-1,000 g/m², more preferably 20 g/m²-800 g/m².

Now, the adhesive skin patch of embodiment 1 is explained. In the adhesive skin patch of embodiment 1, the adhesive layer comprises

the adhesive layer comprises

a thermoplastic elastomer,

(A) a non-volatile hydrocarbon as a liquid component,

one or more kinds selected from the group consisting of (B) an amide solvent and (C) an alcohol solvent, and

solifenacin or a salt thereof. In embodiment 1, the adhesive layer preferably contains all of (A) a non-volatile hydrocarbon oil, (B) an amide solvent and (C) an alcohol solvent as the liquid component. In the absence of a particular description, the explanations of thermoplastic elastomer, liquid component, solifenacin or a salt thereof in embodiment 1 are as mentioned above.

In embodiment 1, while the content of solifenacin or a salt thereof in the adhesive layer is not particularly limited, it is preferably 0.5 wt %-20 wt %, more preferably 1 wt %-17.5 wt %, most preferably 1 wt %-15 wt %, in consideration of the dispersibility in the adhesive layer and the transdermal absorbability.

In embodiment 1, the content of (A) a non-volatile hydrocarbon oil in the liquid component is preferably 15 wt %-90 wt %, more preferably 40 wt %-90 wt %, further preferably 40 wt %-80 wt %, most preferably 40 wt %-60 wt %.

In embodiment 1, to enhance the dispersibility and transdermal absorbability of solifenacin or a salt thereof in the adhesive layer, the total content of one or more kinds selected from the group consisting of (B) an amide solvent and (C) an alcohol solvent is preferably 10 wt %-85 wt %, more preferably 10 wt %-60 wt %, most preferably 20 wt %-60 wt %, of the liquid component.

To enhance the transdermal absorbability of solifenacin or a salt thereof, the liquid component preferably further contains (E) an ester solvent in embodiment 1. In the absence of a particular description, the explanation of (E) an ester solvent in embodiment 1 is as mentioned above.

In embodiment 1, the total content of one or more kinds selected from the group consisting of (B) an amide solvent and (C) an alcohol solvent, and (E) an ester solvent is preferably wt %-85 wt %, more preferably 30 wt %-75 wt %, most preferably 40 wt %-60 wt %, of the liquid component.

In addition, (E) an ester solvent, and one or more kinds selected from the group consisting of (B) an amide solvent and (C) an alcohol solvent are preferably contained at a weight ratio of 1:1-1:5 (weight of (E) an ester solvent:weight of one or more kinds selected from the group consisting of (B) an amide solvent and (C) an alcohol solvent) to enhance the transdermal absorbability improving effect thereof.

To enhance the transdermal absorbability of solifenacin or a salt thereof in embodiment 1, the adhesive layer in the adhesive skin patch of the present invention preferably further contains a surfactant. In the absence of a particular description, the explanation of the surfactant in embodiment 1 is as mentioned above.

In embodiment 1, the content of the surfactant in the adhesive layer is preferably 0.01 wt %-10 wt %, more preferably 0.1 wt %-5 wt %.

In embodiment 1, the content of the liquid component does not generally exceed 1500 parts by weight per 100 parts by weight of the thermoplastic elastomer. In embodiment 1, the content of the liquid component is preferably 320 parts by weight-1000 parts by weight, more preferably 340 parts by weight-700 parts by weight, per 100 parts by weight of the thermoplastic elastomer.

When the content of the thermoplastic elastomer in the adhesive layer is too small, the shape retention of the adhesive layer becomes difficult, and when it is too much, the adhesiveness becomes insufficient. Therefore, the content of the thermoplastic elastomer in the adhesive layer in the adhesive skin patch of embodiment 1 is preferably 5 wt %-24.5 wt %, more preferably 8 wt %-20 wt %, particularly preferably 10 wt %-17.5 wt %. In embodiment 1, the content of the liquid component in the adhesive layer is preferably not less than 50 wt %, more preferably 50 wt %-82 wt %, further preferably 54.5 wt %-79.5 wt %, particularly preferably 60 wt %-75 wt %, most preferably 65 wt %-75 wt %.

The adhesive skin patch of embodiment 1 can exhibit good adhesiveness when the adhesive layer is composed of a thermoplastic elastomer and a liquid component at the above-mentioned contents and content ratios. The adhesive layer may contain a tackifier as necessary. In the absence of a particular description, the explanation of a tackifier in embodiment 1 is as mentioned above.

To decrease skin irritation and the like, the content of the tackifier in the adhesive layer is not more than 10 wt % in embodiment 1. The content is preferably not more than 5 wt %, more preferably not more than 2 wt %, further preferably not more than 1 wt %, and the absence of a tackifier in the adhesive layer is most preferable. In relation to the adhesiveness of the adhesive skin patch, the content of the tackifier is adjusted according to the kind, content and content ratio of the thermoplastic elastomer and liquid component.

In embodiment 1, the adhesive layer may further contain an optional component. In the absence of a particular description, the explanation of the optional component in embodiment 1 is as mentioned above.

To improve stability and further improve transdermal absorbability of a drug, the adhesive layer in embodiment 1 may contain, as an optional component, carboxylic acid and/or a salt thereof or lactone.

Examples of carboxylic acid include aliphatic monocarboxylic acid, alicyclic monocarboxylic acid, aliphatic dicarboxylic acid and the like.

Examples of aliphatic monocarboxylic acid include short chain fatty acids having 2-7 carbon atoms such as acetic acid, butyric acid, hexanoic acid and the like, middle chain fatty acids having 8-11 carbon atoms such as octanoic acid, decanoic acid and the like, long chain fatty acids having 12 or more carbon atoms such as such as myristic acid, stearic acid, isostearic acid, oleic acid and the like, hydroxymonocarboxylic acids such as glycolic acid, lactic acid, 3-hydroxybutyric acid, mandelic acid and the like, alkoxy group-substituted monocarboxylic acids such as methoxyacetic acid and the like, ketomonocarboxylic acids such as levulinic acid and the like, and the like.

Examples of alicyclic monocarboxylic acid include alicyclic monocarboxylic acids having 6-8 carbon atoms such as cyclohexane carboxylic acid and the like.

Examples of aliphatic dicarboxylic acid include sebacic acid, adipic acid, malic acid, maleic acid, fumaric acid and the like.

Preferable carboxylic acid includes, for example, fatty acid having 12 or more carbon atoms and hydroxymonocarboxylic acid, such as myristic acid, stearic acid, isostearic acid, oleic acid and lactic acid. More preferred are oleic acid and lactic acid.

Carboxylic acid may be used as it is or a salt thereof or a mixture with a salt, and preferably used as a salt. Examples of the carboxylate include alkali metal salts such as sodium salt, potassium salt and the like, alkaline earth metal salts such as calcium and the like, amine salt. From the aspects of easy availability and the effect for improving stability and transdermal absorbability, sodium salt is preferably used.

Examples of the lactone include 5-membered ring lactone of ascorbic acid or a salt thereof (e.g., sodium ascorbate), isoascorbic acid or a salt thereof (e.g., sodium isoascorbate) and the like, and the like.

In consideration of the drug stability improving effect and transdermal absorbability improving effect, oleic acid, lactic acid, ascorbic acid, sodium ascorbate, isoascorbic acid or sodium isoascorbate is preferably used as carboxylic acid or a salt thereof or lactone for the adhesive skin patch in embodiment 1.

In embodiment 1, each content of carboxylic acid and/or a salt thereof or lactone in the adhesive layer is not particularly limited. However, from the aspects of transdermal absorbability of a drug and the property of an adhesive skin patch (for example, adhesive property etc.), it is preferably not less than 0.1 mol and not more than 5 mol, more preferably not less than 0.2 mol and not more than 3 mol, per 1 mol of solifenacin.

The explanations on the support and release liner used in embodiment 1, the preparation method of the adhesive skin patch of embodiment 1, the weight per unit area of the adhesive layer after drying and the like are as mentioned above.

Now, the adhesive skin patch of embodiment 2 is explained. In the adhesive skin patch of embodiment 2, the adhesive layer contains

a thermoplastic elastomer, and

(A) a non-volatile hydrocarbon oil, (B) an amide solvent and (D) a liquid organic acid as a liquid component, and

darifenacin or a salt thereof. In the absence of a particular description, explanations on the thermoplastic elastomer, liquid component, and darifenacin or a salt thereof in embodiment 2 are as mentioned above.

In embodiment 2, while the content of darifenacin or a salt thereof in the adhesive layer is not particularly limited, it is preferably 0.5 wt %-20 wt %, more preferably 1 wt %-17.5 wt %, most preferably 1 wt %-15 wt %, in consideration of the dispersibility in the adhesive layer and the transdermal absorbability.

In embodiment 2, the content of (A) a non-volatile hydrocarbon oil in the liquid component is preferably 15 wt %-90 wt %, more preferably 40 wt %-90 wt %, further preferably 40 wt %-80 wt %, most preferably 40 wt %-65 wt %.

In embodiment 2, the total content of (B) an amide solvent and (D) a liquid organic acid is preferably 10 wt %-85 wt %, more preferably 10 wt %-60 wt %, most preferably 20 wt %-60 wt %, of the liquid component, to enhance the dispersibility and transdermal absorbability of darifenacin or a salt thereof in the adhesive layer.

To enhance the transdermal absorbability of darifenacin or a salt thereof, the liquid component preferably further contains (E) an ester solvent in embodiment 2. In the absence of a particular description, the explanation of (E) an ester solvent in embodiment 2 is as mentioned above.

In embodiment 2, the total content of (B) an amide solvent, (D) a liquid organic acid and (E) an ester solvent is preferably 10 wt %-85 wt %, more preferably 30 wt %-75 wt %, most preferably 35 wt %-60 wt %, of the liquid component.

In addition, (E) an ester solvent, and (B) an amide solvent and (D) a liquid organic acid are preferably contained at a weight ratio of 1:1-1:5 (weight of (E) an ester solvent:weight of (B) an amide solvent and (D) a liquid organic acid) to enhance the transdermal absorbability improving effect thereof.

In addition, to enhance the transdermal absorbability of darifenacin or a salt thereof, the adhesive layer in the adhesive skin patch of embodiment 2 preferably further contains a surfactant. In the absence of a particular description, the explanation of the surfactant is as mentioned above.

In embodiment 2, the content of the surfactant in the adhesive layer is preferably 0.01 wt %-10 wt %, more preferably 0.1 wt %-5 wt %.

In embodiment 2, the content of the liquid component does not generally exceed 1500 parts by weight per 100 parts by weight of the thermoplastic elastomer. In embodiment 2, the content of the liquid component is preferably 320 parts by weight-1000 parts by weight, more preferably 340 parts by weight-850 parts by weight, per 100 parts by weight the thermoplastic elastomer.

When the content of the thermoplastic elastomer in the adhesive layer is too small, the shape retention of the adhesive layer becomes difficult, when it is too much, adhesiveness becomes insufficient. Therefore, the content of the thermoplastic elastomer in the adhesive layer in the adhesive skin patch of embodiment 2 is preferably 5 wt %-24.5 wt %, more preferably 8 wt %-21.5 wt %, particularly preferably 10 wt %-12.5 wt %. In embodiment 2, the content of the liquid component in the adhesive layer is preferably not less than 50 wt %, more preferably 50 wt %-87 wt %, further preferably 54.5 wt %-78 wt %, particularly preferably 60 wt %-75 wt %, most preferably 65 wt %-75 wt %.

The adhesive skin patch of embodiment 2 can exhibit good adhesiveness when the adhesive layer is composed of a thermoplastic elastomer and a liquid component at the above-mentioned contents and content ratios. The adhesive layer may contain a tackifier as necessary. In the absence of a particular description, the explanation of the tackifier in embodiment 2 is as mentioned above.

To decrease skin irritation and the like, the content of the tackifier in the adhesive layer is not more than 10 wt % in embodiment 2. The content is preferably not more than 5 wt %, more preferably not more than 2 wt %, further preferably not more than 1 wt %, and the absence of a tackifier in the adhesive layer is most preferable. In relation to the adhesiveness of the adhesive skin patch, the content of the tackifier is adjusted according to the kind, content and content ratio of the thermoplastic elastomer and liquid component.

In embodiment 2, the adhesive layer may further contain an optional component. In the absence of a particular description, the explanation of the optional component in embodiment 2 is as mentioned above.

To improve stability and further improve transdermal absorbability of a drug, the adhesive layer in embodiment 2 may contain, as an optional component, carboxylate or lactone.

Examples of the carboxylate include alkali metal carboxylate such as sodium carboxylate, potassium carboxylate and the like, alkaline earth metal carboxylate such as calcium carboxylate and the like, amine carboxylate. From the aspects of easy availability and the effect for improving stability and transdermal absorbability, sodium carboxylate is preferably used.

In the absence of a particular description, the explanation of carboxylic acid contained in carboxylate is as mentioned above in embodiment 1. Also, the explanation of lactone is as mentioned above.

In consideration of the drug stability improving effect and transdermal absorbability improving effect, sodium oleate, sodium lactate, ascorbic acid, sodium ascorbate, isoascorbic acid or sodium isoascorbate is preferably used as carboxylate or lactone for the adhesive skin patch in embodiment 2.

In embodiment 2, each content of carboxylate or lactone in the adhesive layer is not particularly limited. However, from the aspects of transdermal absorbability of a drug and the property of an adhesive skin patch (for example, adhesive property etc.), it is preferably not less than 0.1 mol and not more than 5 mol, more preferably not less than 0.2 mol and not more than 3 mol, per 1 mol of darifenacin.

The explanations on the support and release liner used in embodiment 2, the preparation method of the adhesive skin patch of embodiment 2, the weight per unit area of the adhesive layer after drying and the like are as mentioned above.

Now, the adhesive skin patch of embodiment 3 is explained. In the adhesive skin patch of embodiment 3, the adhesive layer contains

a thermoplastic elastomer, and

(A) a non-volatile hydrocarbon oil and (B) an amide solvent as a liquid component,

darifenacin or a salt thereof, and

fatty acid salt. In the absence of a particular description, explanations on the thermoplastic elastomer, liquid component, darifenacin or a salt, and fatty acid salt thereof in embodiment 2 are as mentioned above.

In embodiment 3, while the content of darifenacin or a salt thereof in the adhesive layer is not particularly limited, it is preferably 0.5 wt %-20 wt %, more preferably 1 wt %-17.5 wt %, most preferably 1 wt %-15 wt %, in consideration of the dispersibility in the adhesive layer and the transdermal absorbability.

The adhesive layer in embodiment 3 contains a fatty acid salt. In the absence of a particular description, the explanation of the fatty acid salt is as mentioned above.

In embodiment 3, the content of fatty acid salt in the adhesive layer is not particularly limited. However, it is preferably not less than 0.1 mol and not more than 5 mol, more preferably not less than 0.2 mol and not more than 3 mol, per 1 mol of darifenacin. When the content per 1 mol of darifenacin is less than 0.1 mol, a sufficient effect of improving the transdermal absorbability may not be achieved, and when it is more than 5 mol, the preparation property such as adhesive property and the like may be degraded.

In embodiment 3, the content of (A) a non-volatile hydrocarbon oil in the liquid component is preferably 15 wt %-90 wt %, more preferably 40 wt %-90 wt %, further preferably 40 wt %-80 wt %, most preferably 40 wt %-70 wt %.

In embodiment 3, the total content of (B) an amide solvent is preferably 10 wt %-85 wt %, more preferably 10 wt %-60 wt %, most preferably 20 wt %-60 wt %, of the liquid component, to enhance the dispersibility and transdermal absorbability of darifenacin or a salt thereof in the adhesive layer.

To enhance the transdermal absorbability of darifenacin or a salt thereof, the liquid component preferably further contains (E) an ester solvent in embodiment 3. In the absence of a particular description, the explanation of (E) an ester solvent in embodiment 3 is as mentioned above.

In embodiment 3, the total content of (B) an amide solvent and (E) an ester solvent is preferably 10 wt %-85 wt %, more preferably 20 wt %-75 wt %, most preferably 30 wt %-60 wt %, of the liquid component.

In addition, (E) an ester solvent and (B) an amide solvent are preferably contained at a weight ratio of 1:1-1:5 (weight of (E) an ester solvent:weight of (B) an amide solvent) to enhance the transdermal absorbability improving effect thereof.

In embodiment 3, to enhance the transdermal absorbability of darifenacin or a salt thereof, the liquid component preferably further contains (C) an alcohol solvent. In the absence of a particular description, the explanation of (C) an alcohol solvent in embodiment 3 is as mentioned above.

In embodiment 3, the total content of (B) an amide solvent, (E) an ester solvent and (C) an alcohol solvent is preferably 10 wt %-85 wt %, more preferably 20 wt %-75 wt %, most preferably 30 wt %-60 wt %, of the liquid component.

In addition, (E) an ester solvent, and (B) an amide solvent and (C) an alcohol solvent are preferably contained at a weight ratio of 1:1-1:5 (weight of (E) an ester solvent:weight of (B) an amide solvent and (C) an alcohol solvent) to enhance the transdermal absorbability improving effect thereof.

In addition, to enhance the transdermal absorbability of darifenacin or a salt thereof, the adhesive layer in the adhesive skin patch of embodiment 3 preferably further contains a surfactant. In the absence of a particular description, the explanation of the surfactant in embodiment 3 is as mentioned above.

In embodiment 3, the content of the surfactant in the adhesive layer is preferably 0.01 wt %-10 wt %, more preferably 0.1 wt %-5 wt %.

In embodiment 3, the content of the liquid component does not generally exceed 1500 parts by weight per 100 parts by weight of the thermoplastic elastomer. In embodiment 3, the content of the liquid component is preferably 320 parts by weight-1000 parts by weight, more preferably 340 parts by weight-850 parts by weight, per 100 parts by weight the thermoplastic elastomer.

When the content of the thermoplastic elastomer in the adhesive layer is too small, the shape retention of the adhesive layer becomes difficult, and when it is too much, adhesiveness becomes insufficient. Therefore, the thermoplastic elastomer content of the adhesive layer in the adhesive skin patch of embodiment 3 is preferably 5 wt %-24.5 wt %, more preferably 8 wt %-24 wt %, particularly preferably 10 wt %-23.5 wt %. In embodiment 3, the content of the liquid component in the adhesive layer is preferably not less than 50 wt %, more preferably 50 wt %-87 wt %, further preferably 54.5 wt %-78 wt %, particularly preferably 60 wt %-75 wt %, most preferably 65 wt %-75 wt %.

The adhesive skin patch of embodiment 3 can exhibit good adhesiveness, since the adhesive layer is composed of a thermoplastic elastomer and a liquid component at the above-mentioned contents and content ratios. The adhesive layer may contain a tackifier as necessary. In the absence of a particular description, the explanation of the tackifier in embodiment 3 is as mentioned above.

However, to decrease skin irritation and the like, the content of the tackifier in the adhesive layer is not more than wt % in embodiment 3. The content is preferably not more than 5 wt %, more preferably not more than 2 wt %, further preferably not more than 1 wt %, and the absence of a tackifier in the adhesive layer is most preferable. In relation to the adhesiveness of the adhesive skin patch, the content of the tackifier is adjusted according to the kind, content and content ratio of the thermoplastic elastomer and liquid component.

In embodiment 3, the adhesive layer may further contain an optional component. The explanation of the optional component in embodiment 2 is as mentioned above.

In embodiment 3, lactone may be added as an optional component to improve stability and further improve transdermal absorbability of a drug.

In the absence of a particular description, the explanation of the optional component (particularly lactone) in embodiment 3 is as mentioned above.

In embodiment 3, the content of lactone in the adhesive layer is not particularly limited. However, from the aspects of transdermal absorbability of a drug and the property of an adhesive skin patch (for example, adhesive property etc.), it is preferably not less than 0.1 mol and not more than 5 mol, more preferably not less than 0.2 mol and not more than 3 mol, per 1 mol of darifenacin.

The explanations on the support and release liner used in embodiment 3, the preparation method of the adhesive skin patch of embodiment 3, the weight per unit area of the adhesive layer after drying and the like are as mentioned above.

Now, the adhesive skin patch of embodiment 4 explained. In the adhesive skin patch of embodiment 4, the adhesive layer comprises

a thermoplastic elastomer,

(A) a non-volatile hydrocarbon oil as a liquid component,

one or more kinds selected from the group consisting of (B) an amide solvent and (D) a liquid organic acid as a liquid component, and

tolterodine or a salt thereof. In embodiment 4, the adhesive layer preferably contains all of (A) a non-volatile hydrocarbon oil, (B) an amide solvent and (D) a liquid organic acid as the liquid component. In the absence of a particular description, the explanations of thermoplastic elastomer, liquid component, tolterodine or a salt thereof in embodiment 4 are as mentioned above.

In embodiment 4, while the content of tolterodine or a salt thereof in the adhesive layer is not particularly limited, it is preferably 0.5 wt %-20 wt %, more preferably 1 wt %-17.5 wt %, most preferably 1 wt %-15 wt %, in consideration of the dispersibility in the adhesive layer and the transdermal absorbability.

In embodiment 4, the content of (A) a non-volatile hydrocarbon oil in the liquid component is preferably 15 wt %-90 wt %, more preferably 40 wt %-90 wt %, further preferably 40 wt %-80 wt %, most preferably 40 wt %-65 wt %.

In embodiment 4, the total content of one or more kinds selected from the group consisting of (B) an amide solvent and (D) a liquid organic acid is preferably 10 wt %-85 wt %, more preferably 10 wt %-60 wt %, most preferably 20 wt %-60 wt %, of the liquid component, to enhance the dispersibility and transdermal absorbability of tolterodine or a salt thereof in the adhesive layer.

To enhance the transdermal absorbability of tolterodine or a salt thereof, the above-mentioned liquid component preferably further contains (E) an ester solvent in embodiment 4. In the absence of a particular description, the explanation of (E) an ester solvent in embodiment 4 is as mentioned above.

In embodiment 4, the total content of (B) an amide solvent, (D) a liquid organic acid and (E) an ester solvent is preferably 10 wt %-85 wt %, more preferably 30 wt %-85 wt %, most preferably 35 wt %-85 wt %, of the liquid component.

In addition, (E) an ester solvent, and (B) an amide solvent and (D) a liquid organic acid are preferably contained at a weight ratio of 1:1-1:5 (weight of (E) an ester solvent:weight of (B) an amide solvent and (D) a liquid organic acid) to enhance the transdermal absorbability improving effect thereof.

In addition, to enhance the transdermal absorbability of tolterodine or a salt thereof, the adhesive layer in the adhesive skin patch of embodiment 4 preferably further contains a surfactant. In the absence of a particular description, the explanation of the surfactant in embodiment 4 is as mentioned above.

In embodiment 4, the content of the surfactant in the adhesive layer is preferably 0.01 wt %-10 wt %, more preferably 0.1 wt %-5 wt %.

In embodiment 4, the content of the liquid component does not generally exceed 1500 parts by weight per 100 parts by weight of the thermoplastic elastomer. In embodiment 4, the content of the liquid component is preferably 320 parts by weight-1000 parts by weight, more preferably 340 parts by weight-850 parts by weight, per 100 parts by weight the thermoplastic elastomer.

When the content of the thermoplastic elastomer in the adhesive layer is too small, the shape retention of the adhesive layer becomes difficult, when it is too much, adhesiveness becomes insufficient. Therefore, the content of the thermoplastic elastomer in the adhesive layer in the adhesive skin patch of embodiment 4 is preferably 5 wt %-24.5 wt %, more preferably 8 wt %-21.5 wt %, particularly preferably 10 wt %-12.5 wt %. In embodiment 4, the content of the above-mentioned liquid component in the adhesive layer is preferably not less than 50 wt %, more preferably 50 wt %-87 wt %, further preferably 54.5 wt %-86 wt %, particularly preferably 60 wt %-86 wt %, most preferably 65 wt %-86 wt %.

The adhesive skin patch of embodiment 4 can exhibit good adhesiveness when the adhesive layer is composed of a thermoplastic elastomer and a liquid component at the above-mentioned contents and content ratios. The adhesive layer may contain a tackifier as necessary. In the absence of a particular description, the explanation of a tackifier in embodiment 4 is as mentioned above.

To decrease skin irritation and the like, the content of the tackifier in the adhesive layer is not more than 10 wt % in embodiment 4. The content is preferably not more than 5 wt %, more preferably not more than 2 wt %, further preferably not more than 1 wt %, and the absence of a tackifier in the adhesive layer is most preferable. In relation to the adhesiveness of the adhesive skin patch, the content of the tackifier is adjusted according to the kind, content and content ratio of the thermoplastic elastomer and liquid component.

In embodiment 4, the adhesive layer may further contain an optional component. In the absence of a particular description, the explanation of the optional component in embodiment 4 is as mentioned above.

To improve stability and further improve transdermal absorbability of a drug, the adhesive layer in embodiment 4 may contain, as an optional component, carboxylate or lactone.

Examples of the carboxylate include alkali metal carboxylate such as sodium carboxylate, potassium carboxylate and the like, alkaline earth metal carboxylate such as calcium carboxylate and the like, amine carboxylate. From the aspects of easy availability and the effect for improving stability and transdermal absorbability, sodium carboxylate is preferably used.

In the absence of a particular description, the explanation of carboxylic acid contained in carboxylate is as mentioned above in embodiment 1. Also, the explanation of lactone is as mentioned above.

In consideration of the drug stability improving effect and transdermal absorbability improving effect, sodium oleate, sodium lactate, ascorbic acid, sodium ascorbate, isoascorbic acid or sodium isoascorbate is preferably used as carboxylate or lactone for the adhesive skin patch in embodiment 4.

In embodiment 4, the content of carboxylate or lactone in the adhesive layer is not particularly limited. However, from the aspects of transdermal absorbability of a drug and the property of an adhesive skin patch (for example, adhesive property etc.), it is preferably not less than 0.1 mol and not more than 5 mol, more preferably not less than 0.2 mol and not more than 3 mol, per 1 mol of tolterodine.

The explanations on the support and release liner used in embodiment 4, the preparation method of the adhesive skin patch of embodiment 4, the weight per unit area of the adhesive layer after drying and the like are as mentioned above.

### EXAMPLES

The present invention is explained in more detail in the following by referring to Examples and Comparative Examples, which are not to be construed as limitative.

### Examples 1-4

### Preparation of Adhesive Skin Patch Containing Solifenacin Succinate

According to the formulation shown in Table 1, each component constituting the adhesive layer was weighed. First, styrene-isoprene-styrene copolymer (“SIS D1111K”, manufactured by KRATON) was added to liquid paraffin (“KAYDOL”, manufactured by Sonneborn) to give a mixture, and the mixture was dissolved in toluene (31.7 parts by weight per 100 parts by weight of the total content of the adhesive layer components) to prepare a solution. Then, solifenacin succinate and a surfactant were dissolved in the liquid component other than above-mentioned liquid paraffin to prepare a solution. The aforementioned two solutions were mixed by stirring to prepare a coating solution for forming an adhesive layer. “PANACET 810” manufactured by NOF CORPORATION was used as medium-chain triglyceride.

The above-mentioned coating solution was applied to a silicone-treated poly(ethylene terephthalate) (PET) film (release liner), and adjusted such that the weight of the adhesive layer after drying was 100 g/cm². After drying in an oven at 80° C. for 30 min, a PET film (support) was laminated on the surface of the adhesive layer, and the laminate was cut into a size of 15 cm×30 cm to give the object adhesive skin patch.

In preparation of the adhesive skin patch of Example 2, toluene (31.4 parts by weight per 100 parts by weight of the total content of the adhesive layer components) was used.

TABLE 1

| | | Exam- | Exam- | Exam- | Exam- |
|---|---|---|---|---|---|
| | | ple | ple | ple | ple |
| component | | 1 | 2 | 3 | 4 |
| elastomer | styrene-isoprene- | 15.8 | 15.7 | 15.8 | 22.8 |
| | styrene copolymer | | | | |
| liquid component (total) | | 76.2 | 76.6 | 76.2 | 68 |
| (A) non- | liquid paraffin | 36.6 | 36.3 | 36.6 | 38.3 |
| volatile | | | | | |
| hydrocarbon | | | | | |
| oil | | | | | |
| (C) alcohol | propylene glycol | 9.9 | 9.8 | 19.8 | 6.4 |
| solvent | | | | | |
| (B) amide | N- | 9.9 | 9.8 | — | 17.8 |
| solvent | methylpyrrolidone | | | | |
| | crotamiton | 9.9 | 6.9 | 9.9 | — |
| (E) ester | diethyl sebacate | 9.9 | 6.9 | 9.9 | 5.5 |
| solvent | medium-chain | — | 6.9 | — | — |
| | triglyceride | | | | |
| surfactant | sorbitan | 4 | 3.9 | 4 | 0.7 |
| | monolaurate | | | | |
| fatty acid | sodium oleate | — | — | — | 3.2 |
| salt | | | | | |

| | | | | |
|---|---|---|---|---|
| solifenacin succinate | 4 | 3.9 | 4 | 5.5 |

* Numerical values in Table show content (wt %).

### Comparative Example 1

In the formulation of Example 1 of Table 1, instead of the styrene-isoprene-styrene block copolymer, a commercially to available thermosetting pressure-sensitive acrylic adhesive (“Duro tak 87-2194”, manufactured by Henkel, solid content=40 wt %) was weighted such that a solid content thereof was same with that of the thermoplastic elastomer of Example 1 of Table 1, and liquid paraffin was added thereto to prepare a solution. Then, solifenacin succinate and a surfactant were dissolved in the liquid component other than above-mentioned liquid paraffin to prepare a solution. The aforementioned two solutions were mixed by stirring to prepare a coating solution for forming an adhesive layer.

While the above-mentioned coating solution was applied to a silicone-treated PET film (release liner), adjusted such that the weight of the adhesive layer after drying was 100 g/m², and dried in an oven at 80° C. for 60 min, the solution was not cured, and an adhesive skin patch could not be obtained.

### Experimental Example 1

### In Vitro Skin Permeation Test

According to the method described in WO2006/093139, the skin extracted from the abdomen of a male Wister rat (5-week-old) was set on a vertical Franz diffusion cell. Each adhesive skin patch of Examples 1-4 was punched out in a circular shape with a diameter 1.0 cm to give a sample, which was adhered to the rat skin on the diffusion cell (n=3). On the receptor side, using 10% by volume ethanol saline, the content of solifenacin (based on solifenacin succinate) in the receptor solution was measured over time by high performance liquid chromatography (HPLC). The quantification conditions of HPLC are shown below.

### <HPLC Conditions>

HPLC system: high performance liquid chromatograph (LC2010C) manufactured by SHIMADZU CORPORATION

column: ODS, 4.6 mmφ×15 cm, 5 μm

column temperature: 25° C.

mobile phase: buffer/acetonitrile=60/40 (volume ratio)

(buffer; 5.0 mM sodium 1-heptane sulfonate, 1% by volume phosphoric acid)

detection wavelength: 220 nm

flow: 1.0 mL/min

In the aforementioned skin permeation test, according to the content of the solifenacin succinate to be converted to, the amount that permeated through the rat skin was calculated 24 hr after the adhesion. The results are shown in Table 2. From Table 2, each adhesive skin patch of Examples 1-4 of the present invention showed to be superior in skin permeability of the solifenacin succinate.

TABLE 2

| | skin permeation amount 24 hr later |
|---|---|
| sample | (based on solifenacin succinate) (μg/cm²) |
| Example 1 | 777.3 |
| Example 2 | 518.3 |
| Example 3 | 369.4 |
| Example 4 | 746.1 |

### Examples 5-10

### Preparation of Adhesive Skin Patch Containing Darifenacin Hydrobromide

According to the formulation shown in Table 3, each component constituting the adhesive layer was weighed. First, styrene-isoprene-styrene copolymer (“SIS D1111K”, manufactured by KRATON, weight average molecular weight-207,500) was added to liquid paraffin (“KAYDOL”, manufactured by Sonneborn) to give a mixture, and the mixture was dissolved in toluene (41.9 parts by weight per 100 parts by weight of the total content of the adhesive layer components) to prepare a solution. Then, darifenacin hydrobromide and a surfactant were dissolved in the liquid component other than above-mentioned liquid paraffin to prepare a solution. The aforementioned two solutions were mixed by stirring to prepare a coating solution for forming an adhesive layer.

The above-mentioned coating solution was applied to a silicone-treated poly(ethylene terephthalate) (PET) film (release liner), and adjusted such that the weight of the adhesive layer after drying was 100 g/cm². After drying in an oven at 80° C. for 30 min, a PET film (support) was laminated on the surface of the adhesive layer, and the laminate was cut into a size of 15 cm×30 cm to give the object adhesive skin patch.

In preparation of the adhesive skin patch of Examples 6 and 7, toluene (42.9 parts by weight per 100 parts by weight of the total content of the adhesive layer components) was used. In preparation of the adhesive skin patch of Example 8, the drying condition was room temperature for 24 hr. In preparation of the adhesive skin patches of Examples 9 and 10, styrene-isoprene-styrene copolymer (“SIS D1119”, manufactured by KRATON, weight average molecular weight=207,900) was used as an elastomer.

TABLE 3

| component | | Example 5 | Example 6 | Example 7 | Example 8 | Example 9 | Example 10 |
|---|---|---|---|---|---|---|---|
| elastomer | styrene-isoprene- | 10.5 | 10.7 | 10.7 | 10.8 | 23.9 | 21.1 |
| | styrene copolymer | | | | | | |
| liquid component (total) | | 83.8 | 85.7 | 85.7 | 86.5 | 72.5 | 75.3 |
| (A) non-volatile | liquid paraffin | 52.5 | 53.6 | 53.6 | 54.1 | 38.2 | 42.3 |
| hydrocarbon oil | | | | | | | |
| (B) amide solvent | N-methylpyrrolidone | 15.7 | 16.1 | 16.1 | 16.2 | 17.2 | 16.5 |
| | crotamiton | 5.2 | 8.0 | — | 5.4 | 5.7 | 5.5 |
| (D) liquid organic | lactic acid ^{*2} | 5.2 | 8.0 | 8.0 | 5.4 | 5.7 | 5.5 |
| acid | | | | | | | |
| (E) ester solvent | diethyl sebacate | 5.2 | — | 8.0 | 5.4 | 5.7 | 5.5 |
| surfactant | sorbitan | 3.1 | 0.9 | 0.9 | — | 0.8 | 0.7 |
| | monolaurate | | | | | | |

| | | | | | | |
|---|---|---|---|---|---|---|
| darifenacin hydrobromide | 2.6 | 2.7 | 2.7 | 2.7 | 2.7 | 2.7 |

^{*1} Numerical values in Table show content (wt %) in adhesive layer.
^{*2} the Japanese Pharmacopoeia lactic acid

### Comparative Example 2

In the formulation of Example 5 of Table 3, instead of the styrene-isoprene-styrene block copolymer, a commercially available thermosetting pressure-sensitive acrylic adhesive (“Duro tak 87-2194”, manufactured by Henkel, solid content=40 wt %) was weighted such that a solid content thereof was same with that of the thermoplastic elastomer of Example 5 of Table 1, and liquid paraffin was added thereto to prepare a solution. Then, darifenacin hydrobromide and a surfactant were dissolved in the above-mentioned liquid component other than liquid paraffin to prepare a solution. The aforementioned two solutions were mixed by stirring to prepare a coating solution for forming an adhesive layer.

While the above-mentioned coating solution was applied to a silicone-treated PET film (release liner), adjusted such that the weight of the adhesive layer after drying was 100 g/m², and dried in an oven at 80° C. for 60 min, the solution was not cured, and an adhesive skin patch could not be obtained.

### Comparative Example 3

According to the formulation shown in Table 4, each component constituting the adhesive layer was weighed. First, styrene-isoprene-styrene copolymer (“SIS D1111K”, manufactured by KRATON) was added to liquid paraffin (“KAYDOL”, manufactured by Sonneborn) to give a mixture, and the mixture was dissolved in toluene (48.5 parts by weight per 100 parts by weight of the total content of the adhesive layer components) to prepare a solution. Then, darifenacin hydrobromide and a surfactant were dissolved in the liquid component other than above-mentioned liquid paraffin to prepare a solution. The aforementioned two solutions were mixed by stirring to prepare a coating solution for forming an adhesive layer. In the same manner as in Examples 5-10 hereafter, an adhesive skin patch was prepared.

### Comparative Examples 4-6

According to the formulation shown in Table 4, patches were prepared in the same manner as in Comparative Example 3. However, in Comparative Examples 4 and 5, the solubility of darifenacin hydrobromide was poor even when it was mixed by stirring after addition to a liquid component other than liquid paraffin, and an adhesive skin patch containing darifenacin hydrobromide uniformly dispersed therein could not be obtained.

TABLE 4

| | | Comp. | Comp. | Comp. | Comp. |
|---|---|---|---|---|---|
| component | | Ex. 3 | Ex. 4 | Ex. 5 | Ex. 6 |
| elastomer | styrene-isoprene- | 12.1 | 12.1 | 11.3 | 11.3 |
| | styrene copolymer | | | | |
| liquid component (total) | | 84.9 | 84.9 | 85.9 | 85.9 |
| (A) non- | liquid paraffin | 60.6 | 60.6 | 56.6 | 56.6 |
| volatile | | | | | |
| hydrocarbon | | | | | |
| oil | | | | | |
| (B) amide | N- | 18.2 | — | — | 17.0 |
| solvent | methylpyrrolidone | | | | |
| | crotamiton | 6.1 | — | — | 5.7 |
| (C) alcohol | propylene glycol | — | 24.3 | — | — |
| solvent | | | | | |
| (D) liquid | lactic acid | — | — | 22.7 | — |
| organic acid | | | | | |
| (E) ester | diethyl sebacate | — | — | 5.7 | 5.7 |
| solvent | | | | | |
| surfactant | sorbitan | — | — | 0.9 | 0.9 |
| | monolaurate | | | | |

| | | | | |
|---|---|---|---|---|
| darifenacin hydrobromide | 3.0 | 3.0 | 3.0 | 3.0 |

*1; Numerical values in Table show content (wt %) in adhesive layer.
*2; the Japanese Pharmacopoeia lactic acid

### Experimental Example 2

### In Vitro Skin Permeation Test

According to the method described in WO2006/093139, the skin extracted from the abdomen of a male Wister rat (5-week-old) was set on a vertical. Franz diffusion cell. Each adhesive skin patch of Examples 5-10 and Comparative Examples 3 and 6 was punched out in a circular shape with a diameter 1.0 cm to give a sample, which was adhered to the rat skin on the diffusion cell (n=3). On the receptor side, the content of darifenacin hydrobromide in the receptor solution was measured over time using 10% by volume ethanol saline by high performance liquid chromatography (HPLC). The measurement conditions of HPLC are shown below.

### <HPLC Measurement Conditions>

HPLC system: high performance liquid chromatograph (LC2010C) manufactured by SHIMADZU CORPORATION

column: ODS, 4.6 mmφ×15 cm, 5 μm

column temperature: 25° C.

mobile phase: buffer/acetonitrile=70/30 (volume ratio)

(buffer; 5.0 mM sodium 1-heptane sulfonate, 1% by volume phosphoric acid)

detection wavelength: 220 nm

flow: 1.0 mL/min

In the above-mentioned skin permeation test, the amount of darifenacin hydrobromide that permeated through the rat skin was calculated 24 hr after the adhesion. The results are shown in Table 5.

TABLE 5

| | darifenacin hydrobromide skin permeation |
|---|---|
| sample | amount 24 hr after adhesion (μg/cm²) |
| Example 5 | 412.2 |
| Example 6 | 134.2 |
| Example 7 | 249.7 |
| Example 8 | 323.0 |
| Example 9 | 174.0 |
| Example 10 | 176.8 |
| Comparative | 27.4 |
| Example 3 | |
| Comparative | 61.1 |
| Example 6 | |

From Table 5, it was shown that each adhesive skin patch of Examples 5-10 of the present invention is superior in the skin permeability of darifenacin hydrobromide.

On the other hand, the skin permeation amount of darifenacin hydrobromide in the adhesive skin patch of Comparative Example 3 without containing (D) a liquid organic acid was remarkably smaller as compared to the adhesive skin patches of Examples 5-10, and the skin permeability of the drug was clearly inferior.

Similarly, the adhesive skin patch of Comparative Example 6 without containing (D) a liquid organic acid showed low skin permeability of darifenacin hydrobromide even though it contained (E) an ester solvent and a surfactant.

### Examples 11-12

### Preparation of Adhesive Skin Patch Containing Darifenacin Hydrobromide

According to the formulation shown in Table 6, each component constituting the adhesive layer was weighed. First, styrene-isoprene-styrene copolymer (“SIS D1119”, manufactured by KRATON, weight average molecular weight=207,900) was added to liquid paraffin (“KAYDOL”, manufactured by Sonneborn) to give a mixture, and the mixture was dissolved in toluene (50 parts by weight per 100 parts by weight of the total content of the adhesive layer components) to prepare a solution. Then, darifenacin hydrobromide and a surfactant were dissolved in the liquid component other than above-mentioned liquid paraffin to prepare a solution. The aforementioned two solutions were mixed by stirring to prepare a coating solution for forming an adhesive layer.

The above-mentioned coating solution was applied to a silicone-treated poly(ethylene terephthalate) (PET) film (release liner), and adjusted such that the weight of the adhesive layer after drying was 100 g/cm². After drying in an oven at 80° C. for 30 min, a PET film (support) was laminated on the surface of the adhesive layer, and the laminate was cut into a size of 15 cm×30 cm to give the object adhesive skin patch.

TABLE 6

| | | Example | Example |
|---|---|---|---|
| component | | 11 | 12 |
| elastomer | styrene-isoprene- | 22.9 | 23.1 |
| | styrene copolymer | | |
| liquid component (total) | | 70.5 | 71.3 |
| (A) non-volatile | liquid paraffin | 39.0 | 39.5 |
| hydrocarbon oil | | | |
| (B) amide solvent | N- | 17.2 | 18.8 |
| | methylpyrrolidone | | |
| | crotamiton | 5.7 | — |
| (E) ester solvent | diethyl sebacate | 5.7 | 7.2 |
| (C) alcohol | propylene glycol | 2.9 | 5.8 |
| solvent | | | |
| fatty acid salt | sodium oleate | 2.9 | 1.9 |
| surfactant | sorbitan | 0.8 | 0.8 |
| | monolaurate | | |

| | | |
|---|---|---|
| darifenacin hydrobromide | 2.9 | 2.9 |

*1; Numerical values in Table show content (wt %) in adhesive layer.

### Comparative Example 7

In the formulation of Example 11 of Table 6, instead of the styrene-isoprene-styrene block copolymer, a commercially available thermosetting pressure-sensitive acrylic adhesive (“Duro tak 87-2194”, manufactured by Henkel, solid content-40 wt %) was weighted such that a solid content thereof was same with that of the thermoplastic elastomer of Example 11 of Table is 1, and liquid paraffin was added thereto to prepare a solution. Then, darifenacin hydrobromide and a surfactant were dissolved in the liquid component other than above-mentioned liquid paraffin to prepare a solution. The aforementioned two solutions were mixed by stirring to prepare coating solution for forming an adhesive layer.

While the above-mentioned coating solution was applied to a silicone-treated PET film (release liner), adjusted such that the weight of the adhesive layer after drying was 100 g/m², and dried in an oven at 80° C. for 60 min, the solution was not cured, and an adhesive skin patch could not be obtained.

### Comparative Examples 8-9

According to the formulation shown in Table 7, each component constituting the adhesive layer was weighed. First, styrene-isoprene-styrene copolymer (“SIS D1111K”, manufactured by KRATON) was added to liquid paraffin (“KAYDOL”, manufactured by Sonneborn) to give a mixture, and the mixture was dissolved in toluene (48.5 parts by weight per 100 parts by weight of the total content of the adhesive layer components) to prepare a solution. Then, darifenacin hydrobromide and a surfactant were dissolved in the liquid component other than above-mentioned liquid paraffin to prepare a solution. The aforementioned two solutions were mixed by stirring to prepare a coating solution for forming an adhesive layer. Hereafter, adhesive skin patches were prepared in the same manner as in Examples 11-12.

### Comparative Examples 10-11

According to the formulation shown in Table 7, patches were prepared in the same manner as in Comparative Example 8. However, in Comparative Examples 10 and 11, the solubility of darifenacin hydrobromide was poor even when it was mixed by stirring after addition to a liquid component other than liquid paraffin, and an adhesive skin patch containing darifenacin hydrobromide uniformly dispersed therein could not be obtained. Furthermore, sodium oleate was not dissolved in Comparative Example 10, and uniform dispersion state was not afforded.

TABLE 7

| | | Comp. | Comp. | Comp. | Comp. |
|---|---|---|---|---|---|
| component | | Ex. 8 | Ex. 9 | Ex. 10 | Ex. 11 |
| elastomer | styrene-isoprene- | 12.1 | 11.3 | 12.1 | 12.1 |
| | styrene copolymer | | | | |
| liquid component (total) | | 84.9 | 85.9 | 84.9 | 84.9 |
| (A) non- | liquid paraffin | 60.6 | 56.6 | 74.9 | 60.6 |
| volatile | | | | | |
| hydrocarbon | | | | | |
| oil | | | | | |
| (B) amide | N- | 18.2 | 17.0 | — | — |
| solvent | methylpyrrolidone | | | | |
| | crotamiton | 6.1 | 5.7 | — | — |
| (E) ester | diethyl sebacate | — | 5.7 | — | — |
| solvent | | | | | |
| (C) alcohol | propylene glycol | — | — | — | 24.3 |
| solvent | | | | | |
| fatty acid | sodium oleate | — | — | 10 | — |
| salt | | | | | |
| surfactant | sorbitan | — | 0.9 | — | — |
| | monolaurate | | | | |

| | | | | |
|---|---|---|---|---|
| darifenacin hydrobromide | 3.0 | 3.0 | 3.0 | 3.0 |

*1; Numerical values in Table show content (wt %) in adhesive layer.

### Experimental Example 3

### In Vitro Skin Permeation Test

According to the method described in WO2006/093139, the skin extracted from the abdomen of a male Wister rat (5-week-old) was set on a vertical Franz diffusion cell. Each adhesive skin patch of Examples 11-12 and Comparative Examples 8-9 was punched out in a circular shape with a diameter 1.0 cm to give a sample, which was adhered to the rat skin on the diffusion cell (n=3). On the receptor side, the content of darifenacin hydrobromide in the receptor solution was measured over time using 10% by volume ethanol saline by high performance liquid chromatography (HPLC). The measurement conditions of HPLC are shown below.

### <HPLC Measurement Conditions>

HPLC system: high performance liquid chromatograph (LC2010C) manufactured by SHIMADZU CORPORATION

column: ODS, 4.6 mmφ×15 cm, 5 μm

column temperature: 25° C.

mobile phase: buffer/acetonitrile=70/30 (volume ratio)

(buffer; 5.0 mM sodium 1-heptane sulfonate, 1% by volume phosphoric acid)

detection wavelength: 220 nm

flow: 1.0 mL/min

In the above-mentioned skin permeation test, the amount of darifenacin hydrobromide that permeated through the rat skin was calculated 24 hr after the adhesion. The results are shown in Table 8.

TABLE 8

| | darifenacin hydrobromide skin permeation |
|---|---|
| sample | amount 24 hr after adhesion (μg/cm²) |
| Example 11 | 314.7 |
| Example 12 | 441.4 |
| Comparative | 27.4 |
| Example 8 | |
| Comparative | 61.1 |
| Example 9 | |

From Table 8, it was shown that each adhesive skin patch of Examples 11-12 of the present invention is superior in the skin permeability of darifenacin hydrobromide.

On the other hand, the skin permeation amount of darifenacin hydrobromide in the adhesive skin patch of Comparative Example 8 without containing a fatty acid salt was remarkably smaller as compared to the adhesive skin patches of Examples 11-12, and the skin permeability of the drug was clearly inferior.

Similarly, the adhesive skin patch of Comparative Example 9 without containing a fatty acid salt showed low skin permeability of darifenacin hydrobromide even though it contained (E) an ester solvent and a surfactant.

### Examples 13-17

### Preparation of Adhesive Skin Patch Containing Tolterodine Tartrate

According to the formulation shown in Table 9, each component constituting the adhesive layer was weighed. First, styrene-isoprene-styrene copolymer (“SIS D1111K”, manufactured by KRATON, weight average molecular weight=207,500) was added to liquid paraffin (“KAYDOL”, manufactured by Sonneborn) to give a mixture, and the mixture was dissolved in toluene (33.3 parts by weight per 100 parts by weight of the total content of the adhesive layer components) to prepare a solution. Then, tolterodine tartrate and a surfactant were dissolved in the above-mentioned liquid component other than liquid paraffin to prepare a solution. The aforementioned two solutions were mixed by stirring to prepare a coating solution for forming an adhesive layer.

The above-mentioned coating solution was applied to a silicone-treated poly(ethylene terephthalate) (PET) film (release liner), and adjusted such that the weight of the adhesive layer after drying was 100 g/cm². After drying in an oven at 80° C. for 30 min, a PET film (support) was laminated on the surface of the adhesive layer, and the laminate was cut into a size of 15 cm×30 cm to give the object adhesive skin patch.

In the preparation of the adhesive skin patches of Examples 15 and 16, the drying condition was room temperature for 24 hr.

TABLE 9

| component | | Example 13 | Example 14 | Example 15 | Example 16 | Example 17 |
|---|---|---|---|---|---|---|
| elastomer | styrene-isoprene- | 10.7 | 10.7 | 10.8 | 10.8 | 23.6 |
| | styrene copolymer | | | | | |
| liquid component (total) | | 85.2 | 85.2 | 86.1 | 86.1 | 70.9 |
| (A) non-volatile | liquid paraffin | 53.3 | 53.3 | 53.8 | 53.8 | 39.7 |
| hydrocarbon oil | | | | | | |
| (B) amide solvent | N-methylpyrrolidone | 16 | 21.3 | 16.1 | 21.5 | 18.4 |
| | crotamiton | 5.3 | 5.3 | 5.4 | 5.4 | — |
| (C) alcohol solvent | propylene glycol | — | — | — | — | 7.1 |
| (D) liquid organic acid | lactic acid ^{*2} | 5.3 | 5.3 | 5.4 | 5.4 | — |
| (E) ester solvent | diethyl sebacate | 5.3 | — | 5.4 | — | 5.7 |
| surfactant | sorbitan monolaurate | 0.9 | 0.9 | — | — | 0.8 |
| organic acid salt | sodium oleate | — | — | — | — | 1.9 |

| | | | | | |
|---|---|---|---|---|---|
| tolterodine tartrate | 3.2 | 3.2 | 3.2 | 3.2 | 2.8 |

^{*1} Numerical values in Table show content (wt %) in adhesive layer.
^{*2} the Japanese Pharmacopoeia lactic acid

### Comparative Example 12

In the formulation of Example 13 of Table 9, instead of the styrene-isoprene-styrene block copolymer, a commercially available thermosetting pressure-sensitive acrylic adhesive (“Duro tak 87-2194”, manufactured by Henkel, solid content=40 wt %) was weighted such that a solid content thereof was same with that of the thermoplastic elastomer of Example 13 of Table 9, and liquid paraffin was added thereto to prepare a solution. Then, tolterodine tartrate and a surfactant were dissolved in the above-mentioned liquid component other than liquid paraffin to prepare a solution. The aforementioned two solutions were mixed by stirring to prepare a coating solution for forming an adhesive layer.

While the above-mentioned coating solution was applied to a silicone-treated PET film (release liner), adjusted such that the weight of the adhesive layer after drying was 100 g/m², and dried in an oven at 80° C. for 60 min, the solution was not cured, and an adhesive skin patch could not be obtained.

### Comparative Example 13

According to the method of Example 14 described in WO2000/12070, an adhesive layer was prepared according to the formulation shown in Table 10.

TABLE 10

| component | Comparative Example 13 |
|---|---|
| Duro-tak 387-2287 | 88.7 |
| 0.94M aqueous NaOH solution | 3.6 |
| tolterodine tartrate | 7.7 |

*1; Numerical values in Table show content (wt %) in adhesive layer.

### Experimental Example 4

### In Vitro Skin Permeation Test

According to the method described in WO2006/093139, the skin extracted from the abdomen of a male Wister rat (5-week-old) was set on a vertical Franz diffusion cell. Each adhesive skin patch of Examples 13-17 and Comparative Example 13 was punched out in a circular shape with a diameter 1.0 cm to give a sample, which was adhered to the rat skin on the diffusion cell (n=3). On the receptor side, the content of tolterodine tartrate in the receptor solution was measured over time using 10% by volume ethanol saline by high performance liquid chromatography (HPLC). The measurement conditions of HPLC are shown below.

### <HPLC Measurement Conditions>

HPLC system: high performance liquid chromatograph (LC2010C) manufactured by SHIMADZU CORPORATION

column: ODS, 4.6 mmφ×15 cm, 5 μm

column temperature: 25° C.

mobile phase: buffer/acetonitrile=70/30 (volume ratio) (buffer; 5.0 mM sodium 1-heptane sulfonate, 1% by volume phosphoric acid)

detection wavelength: 220 nm

flow: 1.0 mL/min

In the above-mentioned skin permeation test, the amount of tolterodine tartrate that permeated through the rat skin was calculated 24 hr after the adhesion. The results are shown in Table 11.

TABLE 11

| | tolterodine tartrate skin permeation |
|---|---|
| sample | amount 24 hr after adhesion (μg/cm²) |
| Example 13 | 446.7 |
| Example 14 | 137.9 |
| Example 15 | 288.1 |
| Example 16 | 108.5 |
| Example 17 | 333 |
| Comparative | 59.9 |
| Example 13 | |

From Table 11, it was shown that each adhesive skin patch of Examples 13-17 of the present invention is superior in the skin permeability of tolterodine tartrate.

In the meantime, the skin permeation amount of tolterodine tartrate in the adhesive skin patch of Comparative Example 13 prepared according to the method described in WO2000/12070 was markedly low, even though alkali was added to form a tolterodine base, as compared to that of the adhesive skin patches of Example 13-17 containing tolterodine tartrate, and the patch was clearly inferior in the skin permeability of the drug.

### INDUSTRIAL APPLICABILITY

According to the present invention, an adhesive skin patch having sufficient adhesiveness when adhered to the skin, which causes low skin irritation, shows high skin permeability of a therapeutic drug having an anticholinergic activity or a salt thereof for overactive bladder, and is superior in the transdermal absorbability can be provided. Therefore, the adhesive skin patch of the present invention can be utilized as a preparation permitting administration of a therapeutic drug having an anticholinergic activity or a salt thereof for overactive bladder via a route other than oral administration.

This application is based on patent application Nos. 2011-259523, 2012-027739, 2012-0723.54 and 2012-072358 filed in Japan, the contents of which are incorporated in full herein.

1. An adhesive skin patch comprising a support and an adhesive layer containing a drug which is formed on the support, wherein
the adhesive layer comprises
a thermoplastic elastomer,
a liquid component in an amount exceeding 300 parts by weight per 100 parts by weight of the thermoplastic elastomer,
a therapeutic drug having an anticholinergic activity or a salt thereof for overactive bladder as a drug,
(A) a non-volatile hydrocarbon oil as a liquid component, and
one or more kinds selected from the group consisting of (B) an amide solvent, (C) an alcohol solvent and (D) a liquid organic acid as a liquid component, or a fatty acid salt, and optionally comprises a tackifier at a content of not more than 10 wt % of the adhesive layer.

2. The adhesive skin patch according to claim 1, wherein the therapeutic drug having an anticholinergic activity for overactive bladder is one or more kinds selected from the group consisting of solifenacin, darifenacin and tolterodine.

3. The adhesive skin patch according to claim 2, wherein the adhesive layer comprises, as the liquid component, (B) an amide solvent and one or more kinds selected from the group consisting of (C) an alcohol solvent and (D) a liquid organic acid, and
the total content of (B) an amide solvent and one or more kinds selected from the group consisting of (C) an alcohol solvent and (D) a liquid organic acid is 10 wt %-60 wt % of the liquid component.

4. The adhesive skin patch according to claim 3, wherein the adhesive layer comprises, as the liquid component, (B) an amide solvent and (C) an alcohol solvent.

5. The adhesive skin patch according to claim 4, wherein the total content of (B) an amide solvent and (C) an alcohol solvent is 10 wt %-60 wt % of the liquid component.

6. The adhesive skin patch according to claim 3, wherein the adhesive layer further comprises, as the liquid component, (E) an ester solvent.

7. The adhesive skin patch according to claim 3, wherein (A) a non-volatile hydrocarbon oil is liquid paraffin.

8. The adhesive skin patch according to claim 4, wherein the fatty acid salt is a fatty acid salt having 12 or more carbon atoms.

9. The adhesive skin patch according to claim 8, wherein the adhesive layer comprises sodium oleate as at least one of the fatty acid salts.

10. The adhesive skin patch according to claim 3, wherein the adhesive layer further contains a surfactant.

11. The adhesive skin patch according to claim 10, wherein the surfactant is sorbitan fatty acid ester.

12. The adhesive skin patch according to any claim 3, wherein the content of the liquid component in the adhesive layer is not less than 50 wt %.

13. The adhesive skin patch according to claim 3, wherein the thermoplastic elastomer is a styrene block copolymer.

14. The adhesive skin patch according to claim 13, wherein the styrene block copolymer is one or more kinds selected from the group consisting of a styrene-isoprene-styrene block copolymer and a styrene-isoprene block copolymer.

15. The adhesive skin patch according to claim 1, wherein the adhesive layer does not contain a tackifier.

16. The adhesive skin patch according to claim 3, wherein the adhesive layer does not contain a tackifier.
